(19) United States
(12) Patent Application Publication
(10) Pub. No.: US 2002/0094542 A1
(43) Pub. Date: Jul. 18, 2002

(54) Drugs and methods for treating cancer

(76) Inventor: Peter Leskovar, Rosenheim (DE)

Correspondence Address: NEEDLE & ROSENBERG P C, 127 PEACHTREE STREET N E, ATLANTA, GA, 30303-1811, US

(21) Appl. No.: 10/087,252

(22) Filed: Mar. 1, 2002

Related U.S. Application Data
(63) Continuation of application No. 09/302,642, filed on May 3, 1999.
Application No. 09/302,642 is a continuation of application No. 08/808,334, filed on Feb. 28, 1997, now abandoned.
Application No. 08/808,334 is a continuation of application No. 08/160,361, filed on Nov. 29, 1993, now abandoned.
Application No. 08/160,361 is a continuation of application No. 07/989,504, filed on Dec. 11, 1992, now abandoned.
Application No. 07/989,504 is a continuation of application No. 07/449,907, filed on Feb. 13, 1990, now abandoned.
Application No. 07/449,907 is a 371 of international application No. PCT/EP89/00403, filed on Apr. 14, 1989.

(30) Foreign Application Priority Data
Apr. 15, 1998 (DE) P3812605.2

(51) Int. Cl. A61K 39/395; G01N 33/53

(52) U.S. Cl. 435/7.1; 424/144.1

(57) ABSTRACT

A drug is described, containing, in addition to usual carriers-substances, adjuvants and/or additives—a component A, eliminating suppressor-T-cells in a specific or non-specific way, and a component B, activating effector cells. Also provided by the present invention is a method of treating cancer comprising eliminating suppressor cells in the subject, preactivating lymphocytes from the subject ex vivo, and injecting the preactivated lymphocytes into the subject, thereby treating the cancer.

This application is a continuation of and claims priority of U.S. application Ser. No. 09/302,642, filed May 3, 1999 whose status is pending, which is a continuation of Ser. No. 08/808,334, filed Feb. 28, 1997 (now abandoned), which is a continuation of Ser. No. 08/160,361, filed Nov. 29, 1993 (now abandoned), which is a continuation of Ser. No. 07/989,504, filed Dec. 11, 1992, which is a continuation of Ser. No. 07/449,907, filed Feb. 13, 1990, which is a 35 U.S.C. § 371 application of PCT/EP89/00403, filed Apr. 14, 1989, which claims priority from German Application No. P3812605.2, filed Apr. 15, 1988. These applications are hereby incorporated herein in their entirety by this reference.

### BACKGROUND OF THE INVENTION

1. Field of the Invention

The present invention relates to the field of treatment of cancer. Specifically, the present invention provides a composition that affects the immune system of a subject to treat the subject's cancer and a method of treating cancer with the compositions of the invention.

2. Background Art

According to S. A. Rosenberg's LAK-therapy model, the peripheral lymphocytes of the tumor patient are activated polyclonally by a high-dose of IL-2 and reinjected into the patient. The disadvantages of this model are the coactivation of suppressor cells, the non-specific activation of immune cells, and the high costs for IL-2. Conventional tumor therapy (surgery, chemotherapy, radiotherapy, and combined therapy) focuses nearly exclusively on the removal and/or killing of tumor cells and neglects the second, equally important enemy, namely tumor-specific suppressor cells.

It is known that tumor-specific suppressor cells support the establishment of tumor cells by protecting them from the subject's immunological system and are responsible for the unrestricted growth of the tumor. As can be deduced from numerous animal experiments, a permanent cure, not just a temporary remission, is only possible if the suppressor cells are eliminated prior to any immunotherapy. This pre-elimination of suppressor cells is an absolute prerequisite for all forms of immunotherapy. This is true with all kinds of tumors and is independent of their individuality and genetic variability because the tumor-protective suppressor cells, expressing unique, constant, human-specific characteristics and surface structures, are eliminated or inactivated. Thus the substances and substance mixtures of the present invention can be standardized and used for any tumor.

At present, there is practically no hope of eliminating the residual primary tumor and/or micro-metastatic cells by any treatment, so that tumor recurrences are practically “preprogrammed.” Thus, cancer therapies can be based on an active impairment or elimination of residual tumor cells through pre- or reactivated tumoricidal effector cells which possess the fine immunologic tool enabling them to find and recognize the residual tumor cells. As known in the art, activated macrophages and NK-cells are capable of recognizing and killing transformed cells.

### SUMMARY OF THE INVENTION

The present invention provides a method of treating a tumor, wherein the method used does not depend on the type of tumor, comprising two phases. In Phase I (“immune de-repression”), following the surgical removal of the primary tumor, a patient's tumor-protective suppressor cells are eliminated/co-eliminated. In Phase II (“pre- or re-activation of patient's immune response”), the patient's tumoricidal effector cells are pre-activated ex vivo and/or reactivated in vivo. This direct in vivo reactivation is possible due to the pre-elimination of suppressor cells (phase I). Examples of effector cells are macrophages, NK-cells and K/ADCC-cells.

Thus the present invention provides a method for treating cancer in a subject, comprising: (a) eliminating suppressor cells in the subject, (b) preactivating lymphocytes from the subject ex vivo, and (c) injecting the preactivated lymphocytes into the subject, thereby treating the cancer.

### DETAILED DESCRIPTION OF THE INVENTION

It must be noted that, as used in the specification and the appended claims, the singular forms “a,” “an,” and “the” include plural referents unless the context clearly dictates otherwise. Thus, for example, reference to “an antibody” includes multiple copies of the antibody and can also include more than one particular type of antibody.

The present invention provides a method for treating cancer in a subject, comprising: (a) eliminating suppressor cells in the subject, (b) preactivating lymphocytes from the subject ex vivo, and (c) injecting the preactivated lymphocytes into the subject, thereby treating the cancer.

The suppressor cells can be eliminated in a specific way or can be eliminated in a non-specific way. In one embodiment, the suppressor cells can be eliminated by contacting the suppressor cells with antibodies selected from the group consisting of (a) antibodies specific for CD-8 positive suppressor cells, (b) antibodies specific for inducer-suppressor cells, (c) antibodies specific for transducer-suppressor cells, (d) antibodies specific for a subpopulation of radiosensitive suppressor cells, (e) antibodies specific for immunocompetent (pan)-T cells, (f) antibodies specific for (pan)-leukocytes, and (g) polyclonal/polyvalent antibodies or globulins directed against T-cells and thymocytes or lymphocytes.

In another embodiment, the suppressor cells can be eliminated by contacting the suppressor cells with antibodies selected from the group consisting of T cell-specific monoclonal antibodies and T cell-specific polyclonal antibodies. For example, the antibodies can be selected from the group consisting of anti-CD8, anti-CD1, anti-CD3, anti-CD5, anti-CD2 and anti-pan T-antibodies.

In another embodiment of the present invention, the suppressor cells can be eliminated by contacting the suppressor cells with cyclophosphamide and monoclonal or polyclonal antibodies recognizing the suppressor cells.

In another embodiment, the suppressor cells can be eliminated by contacting the suppressor cells with cyclophosphamide and CD-3 or CD-8 positive T-cells.

In the method of the present invention, the lymphocytes of the subject are preactivated by contacting the lymphocytes ex vivo with tumor cells of the subject. A person of skill can practice the methods of the present invention by using the compositions of the invention as described herein.

As used herein, an “antibody (AB),” can be mono- and polyclonal antibodies as well as their fragments, especially their Fab and F(ab′)₂-subunits.

The elimination/co-elimination of suppressor cells can be carried out in a specific or non-specific way. The component A can be: (a) antibodies, specific for the CD8-positive suppressor cells, such as anti-CD8/Leu2/T8(T5)-AB; (b) antibodies, specific for inducer-suppressor cells, such as a combination of subcytolytic concentrations of AB to antigens on the surface of these inducer-suppressor cells, such as Leu3/CD4/T4, Leu8 and Leu18(CD45R); (c) antibodies, specific for transducer-suppressor cells, such as anti-CD1/Leu6/T6-AB or anti-CD38/Leu17/T10-AB; (d) antibodies, specific for a subpopulation of radiosensitive (inducer) suppressor cells, such as anti-CD4/Leu3/T4-AB; (e) antibodies, specific for immunocompetent (pan)-T-cells, such as anti-CD3/Leu4/T3-, anti-CD5/Leu1/T1-, anti-CD2/Leu5b/T11-, partly anti-CD7/Leu9- and anti-Leu7- as well as anti-Leu8-AB; (f) antibodies, specific for(pan)-leukocytes, such as anti-CD45/HLe-1- as well as anti-Leu8-AB; (g) polyclonal/polyvalent antibodies or globulins directed against T-cells and thymocytes and those directed against lymphocytes in general, such as ATS/ATG or ALS/ALG.

In addition, antibodies directed against activated T-cells with a very fast effect can be used. Such antibodies are anti-transferrin-receptor-/anti-T9-AB, anti-CD25/IL-2-receptor/Tac-AB and anti-CD38/Leu17/T10-AB.

The antibodies can be used as combinations, as well, such as the combination of anti-CD8/Leu2/T8-AB with anti-CD4/Leu3/T4-AB, anti-CD3/Leu4/T3-AB, anti-CD5/Leu1/T1-AB and/or anti-CD2/Leu5b/T11-AB. An additional combination is anti-CD8/Leu2/T8-AB and anti-CD45R/Leu18AB and/or anti-CD4/Leu3/T4-AB and/or anti-CD11b-AB.

It is advantageous to use as component A, heteroconjugates, composed of an AB or its Fab/F(ab′)₂-subunit, recognizing suppressor cells specifically or non-specifically, and a molecule that is toxic for suppressor cells or supports their elimination. Examples include, but are not limited to, the following:

(a) RES-dependent: heteroconjugates, composed of AB (IgG-isotype), binding to suppressor cells and Staphylococcus aureus-proteinA (SpA); heteroconjugates, composed of AB and anti-species (mouse)-Ig (with or without complement); heteroconjugates, composed of AB and human, preaggregated IgG (with or without complement); heteroconjugates, composed of AB and xenogeneic proteins.

(b) RES-independent: conjugates, composed of anti-suppressor cell-AB or their Fab/F(ab′)2-subunits and cytotoxins (“immunotoxins”), such as ricin or ricin-alpha chain, abrin, diphtheria-toxin/toxoid, purothionine, modeccine, Pseudomonas-exotoxin, gelonin or pokeweed-toxin; AB-conjugates with radionuclides, such as ^{131/125}I, ¹¹¹In; ⁹⁹Tc or ⁹⁰Yt or with cytotoxic agents, such as doxorubicin, adriamycin, daunomycin, mitomycin C, methotrexate, vinca alkaloids or anthracyclins.

In order to accelerate the RES-elimination of antibody-coated (opsonized) target cells, the xenogenization of antibodies can be achieved by paratope/idiotype-preserving treatment with formaldehyde or glutar(di)aldehyde. Xenogenization can be achieved with all antibodies and their conjugates/heteroconjugates.

To prevent the neutralization of heterologous antibodies (e.g., murine) during the repeated treatment of patients, different procedures can be practiced, such as the use of ABs with the same specificity, raised in different species, or the use of anti-CD4/Leu3/T4-AB and/or anti-B-AB (e.g., anti-CD21-AB) and/or anti-HLA-DR-AB and/or anti-CD25-AB and/or anti-T9-AB. Instead of anti-CD25-AB, a conjugate composed of IL-2 and a cytotoxin can be used. In addition, ABs or their Fab/F(ab′)₂-subunits conjugated to human plasma proteins (primarily to IgG) or to IgG-aggregates and formaldehyde- or glutaraldehyde-pretreated IgG, or to strong tolerogens, such as D-GL, isologous IgG, PEG or PVP, can be used.

Alternatively, conjugates, composed of xenogeneous proteins, especially immunoglobulins and cytotoxins, combined with conjugates, based on the same xenogeneic proteins and tolerogens (D-GL), can be admixed to the component A. In addition, the tolerance to xenogeneic proteins can be induced according to the Chiller/Weigle-method, by injecting highly purified, aggregate-free, heterologous proteins (xenogeneic/murine ABs) prior to the introduction of therapeutic ABs.

Tolerance can also be induced by orally administered xenogeneic proteins, according to the Sulzberger/Chase-principle. A special preparation of the component A contains the antibodies and heteroconjugates and suppressor cell-impairing substances, for example, indomethacin, cimetidine, theophylline, cycloheximide, aminophylline, desoxyguanosine, isoproterenol and histamine-antimetabolites.

Suppressor cell-specific lectins, such as Concanavalin A (ConA) and/or PNA (peanut agglutinin), support the clonal preexpansion of suppressor cells prior to their elimination/coelimination by component A. Cytotoxic agents at low-concentration, primarily alkylating agents (cyclophosphamide) as a part of the component A increase its efficacy.

To inactivate precursor-suppressor cells more efficiently, the component A can be completed with anti-CD1/Leu6/T6-AB or anti-CD38/Leu17/T10-AB. Similar improvement can be achieved by AB, recognizing TdT (terminal deoxynucleotidyl-transferase) on precursor-suppressor cells.

To eliminate/coeliminate the active/activated suppressor cells, conjugates composed of anti-CD25/IL-2(Tac)-receptor-AB, anti-Leu17/T10-AB and/or anti-T9/transferrin-receptor-AB and cytotoxin, or, alternatively, conjugates composed of a ligand, such as IL-2 or transferrin and cytotoxin, can be used. The antibodies and immunotoxins, based on these antibodies, can be combined with conjugates, composed of ligand (IL-2 or transferrin) and cytotoxin, to further increase the efficacy.

The present invention also provides a selective elimination of suppressor cells by a series of suppressor cell-recognizing ABs in subcytolytic concentration which, when given together, contribute to a limiting AB-density on the surface of target cells, critical for their elimination. In this way, suppressor-T-cells can be separated from the cytotoxic T-cells, both being CD8-positive. In addition, the suppressor cell-specific surface structures can be cross-linked by ABs, or more precisely by these structures-recognizing bifunctional heteroconjugates and these “cell labels” on target cells (suppressor cells) conjugated with cytotoxic substances.

The component A can contain combinations of suppressor cell-specific ABs in sub-cytolytic concentration, such as anti-CD8/Leu2/T8(T5)-AB and anti-CD11b/Leu15/CR3-AB or anti-CD4/Leu3/T4-AB and anti-Leu8/AB and anti-CD45R/Leu18-AB as free antibodies or conjugated with cytotoxic agents or radionuclides.

Alternatively, the suppressor cells and their precursors can be “labeled” by Fabor F(ab′)₂-subunits of the mentioned AB-mixtures, followed by the cross-linking with anti-Fab/F(ab′)₂-antibodies. The conjugation of the cross-linking anti-Fab/F(ab′)₂-Ig with cytotoxins, cytotoxic agents or radionuclides supports or replaces the RES-elimination.

Alternatively again, the suppressor cells or their precursors are “labeled” by the corresponding AB-mixture and the following cross-linking by means of anti-species-(mouse)-ABs or their Fab/F(ab′)₂-subunits. For further acceleration of the target cell-lysis, the aforementioned cytotoxic compounds can be post-conjugated.

The next variant is the binding of Fab/F(ab′)₂-subunits of specific ABs to the surface antigens and their cross-linking by a special conjugate, composed of the AB, recognizing the 2nd and the 3rd surface antigen, and anti-Fab/F(ab′)₂-Ig.

The same goal can be achieved by combining biotin-conjugated ABs, directed to different surface antigens on suppressor cells, with conjugates composed of ABs and avidin or anti-biotin. A combination of biotin-labeled mitogenic ABs, such as anti-CD3-AB or anti-TCR-1/WT31-AB, as well as anti-CD2-AB, with avidin- or anti-biotin-conjugated ABs, recognizing surface structures on T-cell subsets, is of special interest. Alternatively, the biotin-labeled mitogenic ABs as well as the avidin- or anti-biotin-conjugated ABs are replaced by the corresponding Fab/F(ab′)₂-subunits.

Analogously, the ABs directed to different suppressor cell-specific surface antigens, when biotin-labeled and kept from mutual interaction, can be easily cross-linked by anti-biotin-conjugated ABs or by bis/tris-avidin-labeled ABs.

Labeled suppressor cells can be eliminated directly in RES-organs, or following the conjugation with cytotoxins, in a RES-independent way. By coupling cytotoxins to antigens or to corresponding anti-idiotypes (and their Fab/F(ab′)₂-subunits, respectively), the tumor-specific, idiotype-bearing T- and B-cells can be eliminated selectively.

Although the tumor-specific suppressor T-cells are coeliminated, it is irrelevant because the coeliminated immunocompetent T- and/or B-cells are soon replaced by maturing precursors. What is important is the essential breaking of the tumor-specific tolerance by depletion/codepletion of tumor-specific suppressor cells. The tumor-specific suppressor cell depletion can be achieved when tumor antigens are known and available.

To prevent the recruitment of tumor-specific suppressor cells, the combination with conjugates, composed of antigen or anti-idiotype (Fab/F(ab′)₂-subunit) and tolerogen (e.g. D-GL), is recommended. An alternative method, based on the immunological network-theory according to Jerne and Wigzell, is the pregeneration of tumor-specific anti-idiotype-subclones of the T- or B-cell lineage, preventing the reinduction of idiotype-bearing suppressor cells following their elimination.

This type of compound is based on immunoconjugates, composed of tumor antigens and of anti-idiotype-antibodies (or their Fab/F(ab′)₂-subunits), respectively, as one part of the conjugate and cytotoxins and/or strong tolerogens (e.g., D-GL) as the second part of the conjugate. These immunoconjugates can be used together with anti-idiotypic ABs or their Fab/F(ab′)₂-subunits and/or with anti-tumor-ABs as the sole, non-suppressive treatment of tumor patients.

The patient's suppressor cells can be preactivated and/or clonally preexpanded, allowing a more radical depletion/codepletion both of active suppressor cells and their precursors (inducer and/or transducer-suppressor cells). By mitogenic lectins (ConA, PNA) and/or low-dosed mitogenic ABs, such as anti-CD3-AB, anti-Ti-AB or anti-CD2-AB and/or IL-2 or IL-3, the clonally preexpanded suppressor cells are post-stimulated, before they are depleted/codepleted by cyclophasphamide, anti-CD8-AB and/or anti-CD25 (anti-T9-, anti-HLA-DR-)AB or the corresponding immunotoxins and/or special conjugates, composed of IL-2 or transferrin and cytotoxins.

The present invention provides a second essential component B of the drug which is responsible for the activation of the effector cells. The activation of effector cells can be carried out in vitro or in vivo.

Both, the in vitro/ex vivo and the in vivo activation of effector cells can be achieved by all substances known to activate immune effector cells, primarily by lymphokines and monokines, such as IL-2, IL-1, M-CSF, GM-CSF, IL-3, IL-2 and M-CSF or GM-CSF, alone or in combination with in vitro preactivated effector cells.

To prevent the growth of suppressor cells in cultures of ex vivo pregenerated (tumoricidal) effector cells, anti-CD8/Leu2/T8-AB, alone or combined with anti-CD4/Leu3/T4-AB and/or anti-CD45R/Leu18-AB, or, alternatively, anti-CD45R/Leu18-AB alone or combined with anti-CD8/Leu2/T8-AB can be added to the cell culture.

Additional preparations contain the previously described ABs (with or without the previously described cytokines and hematological growth hormones, respectively) as well as low-dosed mitogenic ABs, such as anti-CD3/Leu4/T3-AB, anti-TCR-1/WT3l(Ti)-AB, anti-CD2/Leu5/T11-AB and/or low-dosed mitogenic lectins (PHA . . . ). An additional compound in such preparations can be BRMs, such as MDP, MTP-PE and/or inactivated bacteria, such as C. parvum or BCG as well as bacterial lysates.

The component B can include additional compounds such as interferon as well as anti-PGE2 (anti-prostaglandin E2) and/or anti-corticosteroid-ABs. Moreover, a special preparation (component B) includes different heteroconjugates, combined with IL-2 and/or IL-1. These conjugates can have different functions. They can activate the T4or T8-cells in a non-specific/polyclonal or in a tumor-specific/monoclonal way by cross-linking immunorelevant structures on the surface of these cells.

These heteroconjugates mimic the physiologic steps leading to T-cell activation, e.g., the cross-linking of the CD3/T- with the CD4-molecules (via MHC class-II-structures on macrophages) in the case of T4-cells, and the cross-linking of the CD3/Ti- with the CD8-molecules (via MHC class I), respectively. By this active immunoregulatory measure, the critical first step of immune stimulation, i.e., the antigen presentation by macrophages and other antigen presenting cells (APCs), which is impaired in patients with tumors, AIDS and other (retro)viral infections, can be circumvented.

This cross-linking of immunorelevant surface structures by heteroconjugates includes the following membrane molecules on T4- and T8-cells: the cross-linking of CD4 with CD3/Ti, resulting in the activation of T4-cells; the cross-linking of CD8 with CD3/Ti, leading to the activation of T8-cells; the cross-linking of Fc-receptor for IgM with CD3/Ti (activation of T4-(“T_{M}”)-cells, as well as of Fc-receptor for IgG with CD3/Ti (activation of T8-(“T_{G}”)-cells.

The non-specific/polyclonal activation of T4-cells can be achieved by heteroconjugates composed of anti-CD3/Ti-AB, i.e., anti-CD3-AB or anti-Ti-AB and HLA-DR-molecule or with anti-CD4-AB, respectively, as well as heteroconjugates composed of mitogenic lectins, such as PHA and anti-CD4-AB. Instead of ABs, the corresponding Fab/F(ab′)₂-subunits can be used, and instead of lectins, fragments (cleavage products) of lectins can be used.

For the non-specific activation of T8-cells, heteroconjugates, composed of anti-CD8-AB or their subunits and HLA-A-, -B-, -C-molecule, can be used.

The tumor-specific/monoclonal activation of T4-cells can be achieved by heteroconjugates composed of tumor antigen and HLA-DR or anti-CD4-AB, as well as by heteroconjugates composed of anti-idiotype-ABs, mimicking the tumor antigen, and HLA-DR-molecule or, alternatively, by heteroconjugates based on tumor antigen, mimicking anti-idiotype-AB and anti-CD4-AB.

Anti-idiotype-ABs, mimicking tumor antigens, are produced by the immunization of the first animal (e.g., mouse) with tumor antigen, followed by the immunization of a second animal with the Fab/F(ab′)₂-subunit of the tumor-specific immunoglobulin and the isolation of the anti-idiotype-AB from the second animal.

For the tumor-specific activation of T8-cells, heteroconjugates composed of anti-CD8-AB and HLA-A, B, C or, alternatively, heteroconjugates, composed of anti-CD8-AB and lectins or their subunits (cleavage products) can be used.

In addition, the component B can comprise special heteroconjugates, activating T4- and/or T8-cells via the AB-directed interaction of T4- or T8-cells with macrophages (and other APCs). This kind of non-specific/polyclonal or tumor-specific/monoclonal T-cell-activation is based on an immunoregulatively directed T-cell interaction with macrophages or APCs. The T-cell activation mimics the physiologic situation by acceleration of the T:APC-interaction (via specific immunoconjugates). This T:APC-interaction, resulting in T-cell activation, is induced by heteroconjugates (hybrid antibodies), composed of ABs, recognizing membrane structures on macrophages (APCs), and ABs, binding to the CD3/Ti-antigen receptor on T4- and T8-cells.

To activate T4- and/or T8-cells in an non-specific/polyclonal way, heteroconjugates, composed of anti-CD11c/Leu-M5-AB, anti-CD14/Leu-M3-AB or anti-CD15/Leu-M1-AB, and anti-CD5/Leu1/T1-AB or anti-CD2/Leu5b/T11-AB, or, alternatively, heteroconjugates, composed of anti-CD16/Leu11a(b,c)-AB and anti-CD5/Leu1/T1-AB or anti-CD16/Leu11a(b,c)-AB, and anti-CD3/Ti-AB can be used.

The mitogenic anti-CD3/Ti-AB in the above heteroconjugates can be replaced by mitogenic lectins, such as PHA or its subunits (cleavage products).

The tumor-specific/monoclonal activation of T4- and T8-cells can be attained by heteroconjugates, composed of tumor antigen or the corresponding anti-idiotype-AB, and anti-CD11c/LeuM5-AB, anti-CD14/Leu-MB-AB, anti-CD15/Leu-M1-AB or anti-CD16/Leu11a(b,c)-AB. Again, the anti-CD3/Ti-AB can be replaced by mitogenic lectins or their fragments (cleavage products).

These heteroconjugates, designated also as hybrid antibodies, can be synthesized by chemical conjugation techniques, as practiced e.g., in the affinity chromatography for the protein-immobilization or, alternatively, by the hybridomas of the second generation, i.e., by the fusion of hybridoma cells, producing the first Mab as one subunit of the heteroconjugate, with hybridoma cells, producing the second Mab, as the other subunit of the immunoconjugate. The favorable coupling agent is the heterobifunctional compound SPDP (N-succinimidyl-3-(2-pyridyldithio)propionat).

Immune stimulation can be attained by cross-linking the CD3- or the CD2- molecule with the Fc-receptor for IgM (activation of T4-cells) or with the Fc-receptor for IgG (activation of T8-cells).

A non-specific T4-activation (by the component B) can therefore be achieved simply by mitogenic ABs of the IgM-isotype or by heteroconjugates composed of these mitogenic ABs and ABs directed against these mitogenic ABs, belonging to the IgM-isotype. Alternatively, heteroconjugates, composed of the Fab/F(ab′)₂-subunits of the mitogenic AB and the Fe-subunit of the IgM-isotype (Fcμ), can be used. Mitogenic ABs are anti-CD3-AB, anti-CD2-AB, anti-Ti-AB and anti-CD5-AB.

The activation of T8-(instead of T4-)-cells can be achieved analogously by replacing the IgM-isotype with the IgG-isotype in the immunoconjugate. For the specific activation of T4- and T8-cells, the same heteroconjugates as in the case of the non-specific T-cell activation can be used; however, the mitogenic ABs have to be replaced by the antigen or its anti-idiotypic AB. It is advantageous, however, to replace the antigens by their fragments, i.e., enzymatic degradation products. In addition, the anti-idiotypic AB can be replaced by their peptidic fragments, containing 6-15 amino acid-residues, facilitating in this way the cell activation and increasing the spectrum of activated clones, recognizing the particular epitopes. T-cells can be activated additionally by a macrophage-independent cross-linking of the receptors for signal 1 and signal 2 on the surface of T-cells.

Such an APC-independent T4-activation can be attained by special heteroconjugates composed of signal 1 (antigen or mitogen) and of signal 2 (IL-1 or Tp44 or Tp67). Analogously, a T8-activation can be achieved by special immunoconjugates composed of signal 1 (antigen or mitogen) and signal 2 (IL-2). Tp44 and Tp67 can be replaced by their Fab/F(ab′)₂-subunits.

The non-specific (polyclonal) activation of T4-cells can thus be carried out by the component B, consisting of special conjugates, composed of mitogen (mitogenic AB and its Fab/F(ab′)₂-subunit, respectively, or lectin and its subunit, respectively), and IL-1 or Tp44 or Tp67. Both AbsTp44 and Tp67 can replace IL-1 as signal 2. They act synergistically on the T-cell activation by mitogenic ABs (anti-CD3- or anti-Ti-AB). The specific (monoclonal) activation of T4-cells results from the cross-linking of relevant surface structures by conjugates composed of antigen or its anti-idiotypic-AB and IL-1 or Tp44 or Tp67.

A hydrolytic predegradation of antigen or its anti-idiotype (as part of the immunoconjugate) increases the efficiency of activation and clonal expansion. Tp44 and Tp67 can be replaced by their Fab/F(ab′)₂-subunits. In addition, T4-cells can be activated by a combination of IgM and anti-IgM-ABs. For the non-specific (polyclonal) activation of T8-cells, the component B can be composed of a mitogen (mitogenic AB or its Fab/F(ab′)₂-subunit; lectin or its subunits) and IL-2. Advantageously, these heteroconjugates are combined with heteroconjugates in which IL-2 is replaced by IL-1 or Tp44 (Tp67). Alternatively, they can be combined with free IL-1 because the T8-cells, like T4-cells, need IL-1 for their differentiation and proliferation.

A specific (monoclonal) activation of T8-cells can be achieved by heteroconjugates composed of antigen or their anti-idiotypic-AB and IL-2. Anti-idiotypic ABs, pre-enriched by the idiotype-chromatography, i.e., by the passage through a column, loaded with ABs, recognizing the antigen (tumor-antigen), are advantageous subunits of immunoconjugates when compared with non-enriched anti-idiotypic ABs and/or non-processed antigens. Again, combination with heteroconjugates in which IL-2 is replaced by IL-1, as well as mixtures with free IL-1, are advantageous.

Conjugates composed of cell-activating enzymes and cell-recognizing ABs represent a special class of effector cell-stimulating immunoconjugates. Enzymes such as proteases, lipases, mucopolysaccharidases (lysozyme) strongly activate effector cells, such as macrophages and NK-cells. It has been shown that an enzymatic (proteolytic) pretreatment of effector cells increases the activity of macrophages up to 700% and that of NK-cells up to 1300% within 5-10 min. (following a 30-60 min. regeneration phase). Such enzymes are proteases trypsin, chymotrypsin, papain, bromelain, and other proteases, including clinically used urokinase, streptokinase, t-PA (“tissue plasmin activator”), lipases (A1, A2, B, C), mucopolysaccharidases, as well as additional enzymes activating effector cells by changing their cell surface.

Because some of the tested enzymes are secreted by activated macrophages (“exoenzymes”), these hydrolases cause not only the degradation of target cells but additionally the autoactivation of macrophages as well as the coactivation of cooperating effector cells (e.g., NK-cells).

To activate the effector cell more selectively, heteroconjugates composed of enzymes (proteases, lipases, mucopolysaccharidases, especially lysozyme and neuraminidase/sialidase) and effector cell-specific Abs can be used. These ABs can be specific for macrophages (e.g., CD11c/CD14/CD15), NK-cells (CD16, Leu19) or CTLs (CD3/CD2/CD5). To prevent the coactivation of suppressor-monocytes, the macrophage/monocyte-specific ABs (e.g., CD11c/CD14) can be conjugated with prostaglandin-inhibitors (e.g., indomethacin, aspirin).

To prevent the neutralization of the macrophage-secreted proteases by alpha2-macroglobulin and alpha1-proteinase-inhibitor, which are both secreted by activated macrophages, heteroconjugates composed of the same anti-macrophage/monocyte-ABs and anti-alpha2-macroglobulin, anti-alpha1-proteinase-inhibitor, anti-trypsin and/or anti-chymotrypsin can be used.

A direct macrophage-activation can be achieved by a special conjugate, consisting of anti-macrophage/monocyte-ABs and endotoxin or gamma-interferon. Since the antimicrobial and tumoricidal activity of macrophages is based on the oxidation via peroxide (O₂⁻), hydrogen peroxide (H₂O₂), hydroxyl-radical (OH.) and atomic O₂ (¹O₂) (Klebanoff), heteroconjugates, composed of anti-macrophage/monocyte-ABs and (myelo)peroxidase and/or catalase are recommended.

The efficacy of such conjugates can be increased by coupling anti-glutathion-peroxidase and/or anti-glutathion-reductase to anti-macrophage/monocyte-ABs. The addition of halides, such as Cl⁻ or I⁻, is advantageous. The conjugate of the same ABs with dibutyryl-cGMP activates the target cell via Ca²+-influx. This conjugate can be combined with another conjugate composed of AB and ionophore (e.g., lonophore A 23187), which mobilizes the intracellular Ca²⁺ (calcisomes).

Conjugates, composed of anti-macrophage/monocyte-AB and theophylline, theobromine, caffeine, catecholamine (e.g., epinephrine, norepinephrine and isoproterenol) and additional CAMP-stimulators can be used for a selective effector cell-activation as part of component B.

Macrophages can further be activated by conjugates composed of anti-macrophage/monocyte-ABs and complement-subunits, such as C1, C2, C3, C4, C5, C3bi, C5a. Analogous to mast cells, which are activated by IgE and anti-IgE, or analogous to B-cells, which are activated by anti-IgM-ABs, macrophages can be activated by IgG and anti-IgG-conjugates. Because of the essentially facilitated phagocytosis, conjugates consisting of anti-macrophage/monocyte-ABs and fibronectin and/or cyto/heterophilic ABs can be recommended.

The same type of conjugates, described above in association with the activation of macrophages, can be introduced in order to activate NK-cells and CTLs. In these conjugates, the anti-macrophage-ABs must be replaced by anti-NK-ABs and anti-CTL-ABs, respectively.

The next class of specific conjugates comprises those composed of compounds able to activate cells directly or to mimic such direct cell-activators and effector-specific ABs. During the cell activation, two active metabolites, derived from the membrane-associated inositol, are released, namely, the inositol triphosphate (IP₃) and the diacylglycerol (DAG). The first metabolite increases the cytoplasmic Ca²+via Ca²⁺-mobilization from the ER-deposit (calcisome). The DAG, on the other hand, activates the enzyme protein kinase C (PKC). For the T-cell activation, an additional signal (signal 2, i.e., IL-1) is necessary. Ca-ionophores, increasing the cytoplasmatic Ca²⁺-level directly, as well as the phorbol esters, activating directly the PKC lead to cell activation which is signal 2 (IL-1) independent because phorbol ester in addition mediates the transmembranal signal-transmission. To induce a selective effector cellactivation, conjugates composed of IP3, DAG, Ca²⁺-ionophore and/or phorbol ester and anti-effector cell-AB, are recommended.

Stimulation of effector cells can also be achieved by coupling lysophosphatides on effector cell-specific ABs; the lysophospholipids are conjugated to effector cell-specific ABs by means of MPB-PE (N-(p-meleimidophenyl)butyryl-phosphatidylethanolamin).

As described with suppressor cells, the CTLs can be “labeled” with specific ABs or their mixture and activated by coupling mitogenic ABs or mitogenic lectins. The innovative drug of the present invention contains the component A, with anti-CD11b-AB, mediating the elimination of suppressor cells, and/or with anti-CD45R-AB, mediating the depletion of inducer-suppressor cells, and component B, composed of mitogenic AB (low-dose anti-CD3- or anti-TCR-1/WT31-AB) or mitogenic lectins (PHA), conjugated to anti-Leu19-AB, mediating the selective activation of cytotoxic T-(and NK)-cells, immediately following the preelimination of suppressor by the component A.

A selective stimulation of effector cells can also be achieved by cross-linking murine antibodies on the surface of effector cells by anti-mouse-ABs or their Fab/F(ab′)₂-subunits; the cell-bound AB must, however, have mitogenic properties (e.g., anti-CD3-AB). Mitogenic ABs can be replaced by mitogenic lectins or their proteolytic cleavage products. Biotin:Avidin-conjugates or biotin:anti-biotin-Ig-conjugates can be replaced by Ag:Ab (antigen:antibody) conjugates; such conjugates can be composed of (inactivated) enzymes and antibodies to these enzymes, e.g., inactivated G-6-PDH and anti-G-(-PDH-Ig, or inactivated (horse radish) peroxidase and anti-peroxidase-Ig. The cross-linking can also be achieved by plasma proteins, conjugated to first AB, and those, coupled to second AB, e.g., conjugates consisting of the first AB (Mab) and human IgG or IgM or albumin or (inactivated) ceruloplasmin, which are combined with the conjugates composed of the second AB (Mab) and the antigen-specific AB or gamma-globulin, respectively.

According to an alternative variant, both cooperating cells—macrophages (APC) and T4- or T8-cells—are forced to interact by heteroconjugates composed of anti-CS15/Leu-M1-AB, anti-CD14/Leu-MS-AB or anti-CD11c/Leu-M5-AB, and anti-CD5/Leu1/T1-AB or anti-CD2/Leu5b/T11-AB. These heteroconjugates can be combined with antigen, e.g., tumor-antigen, its anti-idiotypic-AB and/or with anti-CD3/Leu4/T3-AB or anti-TCR-1/WT31-AB, respectively, to further stimulate T4- and T8-cells.

To produce an antigen-specific immune stimulation, a combination of heteroconjugates composed of antigen or its anti-idiotypic AB and the corresponding (antigen-specific) Ig of the IgM-isotype, with heteroconjugates composed of antigen or its anti-idiotype-AB and anti-CD4/Leu3/T4-AB (for the T4-stimulation) or anti-CD8/Leu2/T8-AB (for T8-stimulation) can be used. Analogously, the non-specific/polyclonal T4- and T8-activation can be achieved by combining heteroconjugates composed of anti-CD3/Leu4/T3-AB or anti-TCR-1/WT31-AB and Ig (of the IgM-isotype), directed to these ABs, with anti-CD3/Leu4/T3-AB or anti-TCR1/WT31-AB.

A replacement of the antigen (tumor-antigen) in the above described conjugates by antigen-fragments, resulting from an epitope-protecting enzymatic or chemical precleavage of the corresponding antigen (tumor-antigen) to 6-10 peptide- or monosaccharide-fragments is associated with an increased immunostimulatory efficiency of the conjugates. These single antigen-subunits have to be coupled to the common second component of the original heteroconjugate. In this way, a higher number of T4- or T8-clones, respectively, are mobilized and immediately (via T4-cells) additional B-cells are activated to plasma cells. This enzymatic “pre-digestion” mimics the processing of the antigen by macrophages.

If the component B is used in vitro, i.e., for the ex vivo treatment of a patient's lymphocytes, a higher specificity can be obtained by pre-activating the patient's effector cells in the presence of the inactivated, unchanged or chemically/enzymatically pretreated tumor cells (membrane fragments, 3 M Kcl-tumor-extracts). A coactivation of suppressor cells can be prevented by a hydrolase-pretreatment of effector cells. In the case that this pretreatment would not be effective enough, the coactivated suppressor cells and/or their precursors (inducer-suppressor cells) can be depleted as described above.

The effector cell-activation by IL-2 can be supported by hydrolases. The in vivo action of the component B can be mediated by oral enzyme preparations, which act proteolytically after their resorption, as can be deduced from their therapeutical use in patients with autoimmune diseases (degradation of immune complexes).

The effector cells can also be activated by fusogenic substances at sub-fusogenic concentrations, as well as by substances activating effector cells (a) by their influence on the electrokinetic (zeta) potential (b) by their influence on the hydration-enthalpy of the membrane-lipids, and/or (c) by their influence on the dielectric constant (DK) of the medium. These substances intensify the effector cell:target cell (E:T)-contact, resulting in the activation of the effector cell, as well as mobilization of additional lymphocyte-clones, primarily T4- and T8-cells.

Examples of such substances include, but are not limited to, polyethyleneglycol (PEG), polyvinylpyrrolidone (PVP), those polymers with medium and higher molecular weights, polyvinylalcohol (PVA), polyvinylacetate (PVAC, hydroxyethyl-starch, dextran and its derivatives, polyalcohols (polyols), such as mannitol and sorbitol, fatty acids and their derivatives, and substances with fusogenic and/or with physicochemical properties in general.

The use of PVP and PEG is advantageous especially for the in vitro activation, because these polymers are excellent cryoprotectants. They thus can be used alone or, preferentially, combined with DMSO (8-20%) for the freezing and preservation of cells, e.g., leukocytes, as well as for the preservation of activity of preactivated effector cells. The preactivated state of effector cells could only be preserved when the cells were frozen in the presence of PVP or PEG added to DMSO.

It is generally advantageous to replace or to complete antigen-based conjugates by those, containing enzymatically or chemically precleaved antigen-fragments and/or antigen-mimicking anti-idiotypic ABs (Mabs) or their Fab/F(ab′)₂-subunits, instead of whole antigens. The antigen-fragments in these heteroconjugates should be composed of oligopeptides with 6-10 amino acid-residues, or oligosaccharides with 6-10 monosaccharide-units, or corresponding oligoglycopeptides, respectively, depending on the molecular structure of antigens (tumor-antigens: TATA, TSTA, TSA . . . ). This type of effector cell-activation mimics the physiological situation, where antigens, including tumor antigens, are first “processed,” i.e., cleaved to fragments (e.g., peptides) by (lysosomal) hydrolases of macrophages and other APCs and thereafter, in context with MHA II, “presented” to helper-T4-cells which become activated in the presence of signal 2 (monokine IL-1).

To further improve the efficacy of the described heteroconjugates, the component B can contain two special Mabs (or their Fab/F(ab′)₂-subunits), i.e., the Tp67-Mab (corresponding to the CD5/Leu1/T1-Mab) and/or Tp44-Mab as well as the “signal 2” of helper-T-activation. These Mabs show, like IL-1, a synergistic action on T4-cells. They can be used both for the non-specific/polyclonal, as well as for the (tumor)specific T-cell-activation. They can serve as adjuvants; they are bound to the surface of T-cells and activate selectively those T-cells which recognize tumor antigens. Thus, an antigen-specific immune stimulation can be achieved even if the structure of tumor antigen is not known.

In addition, heteroconjugates can be synthesized wherein one component is Tp67 or Tp44 (or their subunit) and the other component is either anti-CD3- or anti-Ti-receptor-Mab (or its Fab/F(ab′)₂-subunit). These heteroconjugates induce a polyclonal T-cell-activation. In contrast, heteroconjugates, in which the anti-CD3- or anti-Ti-receptor-Mab is replaced by (tumor)antigen (or its fragments) and/or by anti-idiotypic ABs, mimicking this antigen (or their Fab/F(ab′)₂-subunit), induce a monoclonal, antigen-specific T-cell-activation.

Depending on the situation, the AB as a component of a particular heteroconjugate must either induce the elimination of the target cell (e.g., depletion of suppressor cells) or conversely mediate its selective activation (e.g., specific and non-specific T-cell-activation). This can be achieved by use of the correct isotype and/or the Fab/F(ab′)₂-subunit instead of the whole Ig-molecule. An example of such a discrimination between RES-eliminated and non-eliminated isotypes is the (rat) anti-Lyt1-AB in murine model, corresponding to the anti-CD5-AB in humans. The Ig2b-isotype leads to the lysis and RES-depletion of murine Lyt⁺-cells, whereas the Ig2a-isotype with the same specificity results in the activation of Lyt⁺-cells without cytolysis and cell-depletion, respectively.

In addition to the components A and B, the drug can contain further heteroconjugates, composed of different cytokines, such as IL-1, IL-2, IL-2, G-CSF and GM-CSF. Further, heteroconjugates composed of IL-1 and IL-2 or IL-3 or G(GM)CSF and those, composed of IL-2 and IL-3 or M-CSF or GM-CSF, respectively, can be recommended. Conjugates, consisting of interferon(s) and IL-1 or IL-2 or IL-3 or M-CSF or GM-CSF are of interest as well, as are those composed of interferon(s) and mitogens (mitogenic antibodies or mitogenic lectins and their subunits, respectively (polyclonal activation). Replacement of mitogens by antigen or its anti-idiotypic AB in the corresponding heteroconjugates results in a monoclonal T-cell-activation. The special advantage of an interferon-containing conjugate is the increased antigenicity of the target cell, caused by the post-expression of MHC II (HLA-DR)- and/or MHC I (HLA-A,B,C)-antigens.

The “acquired immunodeficiency syndrome” (AIDS) is subject of numerous reports, published in specialized journals, such as “AIDS-Forschung” (AIFO) (editor: R. Hehlmann; Schulz-Verlag, München) or “AIDS Research and Human Retroviruses” (editor: D. P. Bolognesi; M. A. Liebert Publishers, New York), as well as in scientific journals, such as “Nature” and “Science.” An overview is given in the “Basic & Clinical Immunology” (P. Stites, J. D. Stobo, J. V. Wells, editors; Appleton & Lange Publishers, Norwalk, 1987). Therapeutic approaches are disclosed within the review article “Strategies for antiviral therapy in AIDS,” by H. Mitsuya and S. Broder, published in Nature 325:773 (1987).

There exist no drugs which are able to cure AIDS, and no vaccines for a successful prophylactic anti-AIDS-vaccination. Most of the currently used drugs, for example, AZT, focus on the inhibition of the HIV-replication.

Patients with AIDS, ARC and/or LAS show a fatal immunodeficiency and a fatal immunosuppressive constellation which are worse than in tumor patients and facilitate the spread of normally harmless opportunistic inflections, as well as the establishment of normally rare tumors (Kaposi's sarcoma). The compositions of the present invention prevent this basic failure and contribute to the remission by eliminating HIV-protecting suppressor cells and reactivating the patient's immune response because the HIV virus is not only inhibited in its replication but additionally eliminated.

The most difficult problem is the HIV-infection of nerve and brain cells. As with residual tumor cells, these residual HIV can be attacked by effector cells, as soon as the protective suppressor cells have been eliminated or essentially reduced and the effector cells pre- or reactivated.

Herewith, a critical turn can occur, induced by a quantitative change in the ratio of leukocyte-subpopulations (e.g., the T4/T8-ratio or the ratio of CTLs to suppressor cells).

The composition of the present invention acts in 3 phases:

(a) phase I₀: elimination of HIV, without or with the component C;

(b) phase I: elimination/co elimination of HIV-protective suppressor cells by means of the component A

(c) phase II: in vitro preactivation or in vivo reactivation of HIV-controlling and eliminating effector cells with component B.

Phase I₀ and phase I can partly be accompli shed by the sole component A; however, the use of the third component C for the phase I₀ is necessary. Both phases I analogous to phases I and II described in the tumor treatment. Therefore, only the differences in treatment of AIDS patients, as compared with the treatment of tumor patients, will be described below, and the common steps of therapy will not be repeated. They have been described in all details as phase I and II in tumor patients above.

The potential HIV-host-cells (T4-cells and to some extent macrophages/monocytes, B-cells, T8-cells) have to be opsonized by specific ABs and thereafter RES-eliminated, independent of their HIV-infection and of the membrane expression of viral structures (HIV env-glycopeptides).

The present invention provides compositions that can be combined with anti-drugs (AZT and other drugs, e.g., the virustatic agents and other AIDS drugs, cited in Mitsuya and Broder above.

The elimination of HIV in T4-cells, as well as in T8-cells, B-cells and macrophages/monocytes, can be accomplished by following ABs and AB-combinations which are partly preincluded in the component A (see above): anti-CD4/Leu3/T4-AB and/or anti-CD3/Leu4/T3-AB, combined with anti-CD8/Leu2/T8(T5)-AB, anti-CD19/Leu12-AB, anti-CD22Leu14-AB, anti-CD20/Leu16-AB, anti-CD21/CR2-AB, anti-CD15/LeuM1-AB, anti-CD14/LeuM3-AB or anti-CD11c/LeuM5-AB, as well as anti-Leukocyte/HLe-1-AB (a pan-leukocyte-AB) or anti-lymphocyte/thymocyte-serum (or the corresponding globulins) of the type ALS/ALG or ATS/ATG. These ABs can be additionally combined with ABs, recognizing viral env-glycopeptides, e.g., anti-gp 120/160-Ig, as well as with ABs, recognizing the nuclear material of HIV-infected cells. This nuclear material can be standardized by immunization of animals with HIV-infected H9- or ATH8-cells, pre-lysed with surfactants, ultrasound or shock-freezing.

The elimination of HIV-host cells can be intensified analogously to the depletion of suppressor cells (phase I of tumor treatment, see above). The efficiency of depletion of HIV-host cells can further be increased by a repeated injection of allogeneic T-cells which have been allopreactivated in vitro by a type of MLC/MLR to become cytotoxic for the patient's T-cells.

Since the elimination of HIV-protective suppressor cells corresponds completely to that of tumor-specific suppressor cells, the component A can be composed of the same active substances. As already mentioned, the phase I₀ and phase I can be used together, as “phase I”; the present ABs and AB-combinations fulfill both functions, the elimination of HIV and of suppressors. This is valid for all substances and mixtures (ABs and AB-combinations), described above (phase I₀), except for the use of anti-CD4/Leu3/T4-AB, both as mono-treatment or combined treatment (with anti-B-cell- and/or anti-macrophage/monocyte-AB).

The same is true with the phase II-treatment, i.e., the in vitro preactivation or direct in vivo reactivation of effector cells with the component B which can be used in the composition(s), described above. The preactivation of autologous effector cells in vitro can, however, be supported by virustatic agents, such as AZT; the AZT-concentration can be higher than the therapeutic one.

To increase the specificity of pregenerated effector cells, inactivated, chemically or enzymatically modified, attenuated HIV, preferentially fixed to H9- or ATH 8-cell surface, can be added to the system, both in vitro and in vivo. To mobilize additional, cross-reacting lymphocyte-clones, the inactivated, partially modified HIV can be combined with SIV, especially those showing only 10-30% homology with HIV. The inactivated HIV and SIV can be fixed on different, or alternatively, the same—but chemically or enzymatically “xenogenized”—carrier cells.

To induce an antigen-specific immunization against opportunistic infections in AIDS/ARC/LAS-patients during the immune reconstitution in the phase II, the patient's effector cells, e.g., CTLs can be coincubated with a standardizable antigen-mixture (e.g., lysate of the representatives of typical opportunistic infections), independent of preexistent or absent opportunistic infection.

The same details, concerning tumor antigen, its fragments (cleavage products), as well as the corresponding anti-idiotypic Ig, as part of the (hetero)conjugates, are valid in the case of HIV-antigens and their immunologically relevant representatives, the env(gp120/160/110/130)-glycoproteins.

In AIDS-patients, both antibodies and cytotoxic T-cells directed not only to env- but also to gag- and pol-gen products, as well as antibodies against viral enzymes and regulatory proteins, have been detected. Therefore, heteroconjugates, composed of viral antigens or their fragments or their anti-idiotypic Ig or their Fab/F(ab′)₂-subunits, respectively, and anti-CD3-, or anti-Ti-antigen-receptor, or CD2 or Tp67(CD5) or Tp44, respectively, can be used. The term “viral antigens” comprises all antigens, including the core-antigens of HIV, the env-glycopeptides being, however, of central interest.

All other (hetero)conjugates and techniques for T-cell-activation are analogous to those described in tumor patients. The only difference is the replacement of tumor-antigen, designated as “antigen,” and its anti-idiotypic Ig in the heteroconjugates by viral antigens, especially the env-glycopeptides and their anti-idiotypes.

A HIV-specific immunization can be achieved by viral peptides, coupled to different, non-crossreacting carrier-proteins.

The principle is the breaking of a possible residual tolerance to HIV-structures, secondary to the suppressor cell-elimination, by coupling HIV-antigens, primarily the env-glycopeptides, to strongly immunogenic carrier-proteins; the cross-reactivity of the global molecule (the same haptenic group on different carriers) should account for 15-25% and should not exceed 75%. The viral antigens can be replaced by the corresponding anti-idiotypes. These haptene:carrier-conjugates can be standardized. They can be combined with antibodies, recognizing the tolerance-susceptible CD1(T6)- and CD38(T10)-positive T-cell precursors. Such conjugates can be used both for the prophylaxis and for the therapy of AIDS/ARC/LAS.

The low-molecular weight CD4-fragments and fragments of the gp 120-(gp 160)- molecule, containing 10-20 amino acid-residues, inhibit the interaction of HIV with the CD4-receptor, without being immunogenic. Herewith, a neutralization of the therapeutically used CD4-molecule can be prevented. These fragments can be produced biotechnologically. This inhibition is based on the analogous principle as the elution of macromolecular ligands by mono- or oligomers in affinity chromatography, or as the inhibition of lectin-binding to glycoproteins in the presence of specific monosaccharides.

Such gp 120-(gp 160)-fragments can be recommended both for the prophylaxis as well as for the therapy of AIDS/ARS/LAS-patients.

The HIV:CD4-interaction can also be inhibited by low-molecular weight-, non-immunogenic HLA-DR-subunits. The HLA-DR-molecule is the direct ligand of the CD4-receptor. Its low-molecular weight-, non-immunogenic fragments compete with HIV for the CD4-receptor. A preenrichment of HLA-DR-fragment, interacting with CD4, can be achieved by a CD4-chromatography. Between gp 120 (gp 160) and HLA-DR, there must exist homologies, since both interact with the CD4-molecule; analogously, a homology between gp41 and HLA-DR is discussed. Therefore, these HLA-DR-fragments can be considered in the prophylaxis and therapy of AIDS/ARC/LAS.

The Fab/F(ab′)₂-subunits of anti-CD4-ABs compete with HIV for the CD4-receptor. Due to their lower molecular weight (50 kD), they are less immunogenic and can pass the brain-blood-barrier. They can be used prophylactically and therapeutically.

A binding of free HIV and free env-glycopeptides in plasma can be achieved by sub-dosed anti-HLA-DR-ABs. Sub-dosed anti-HLA-DR-ABs and/or their Fab/F(ab′)₂-subunits can “neutralize” free HIV and free env-glycopeptides in plasma, due to the homologies between HLA-DR and env-glycopeptides (gp 120 and/or gp 41). Since homologies between HIV- and other retroviral env-glycopeptides on the one hand and IL-2 on the other have been reported and the immunosuppressive action of retroviruses originates in this homology, a combination of sub-dosed anti-HLA-DR-ABs and IL-2 is advantageous. The soluble form of HLA-DR is, contrary to the membrane-associated form, immunosuppressive.

In a preferential preparation, the above described heteroconjugates, especially those which activate T-cells independently of APCS, are combined with IL-1 and/or IL-2. For an additional increase of efficiency, mitogenic ABs and/or lectins (PHA), as well as BRMs can be admixed.

Syncytia formation can be prevented by conjugates, composed of antiviral ABs and strongly polar, acidic or basic groups, due to the high electrokinetic(zeta)potential of conjugate-loaded target cells.

Heteroconjugates, composed of HIV-specific ABs and mitogenic ABs (anti-CD3-, anti-Ti-, anti-CD2-, anti-CD5-ABs) unite a high specificity with a polyclonal activation; the binding of such heteroconjugates to viral membrane structures of infected cells renders possible the binding and activation of T-cells in an antigen-non-specific and MHC-nonrestricted way.

The outer and inner lipid membrane of HIV can be damaged by hydrophilic polymers, showing a partial lipophilic character of the molecule. Since experiments have shown an increased solubility in watery solution of polymers, such as PVP and PEG, polymers of this kind—some of them belonging to the class of plasma expanders (PVP, hydroxyethylstarch)—could be used in vivo to inactivate plasma-HIV.

It is known that the efficiency of elimination of circulating immunocomplexes (CIC) strongly depends on their composition, as well as their molecular weight. Therefore, the viral antigen in CIC should be replaced by corresponding anti-idiotypic AB; its Fc-subunit could accelerate the phagocytosis by macro- and microphages. An additional effect could be achieved by the heat-aggregation or chemical predenaturation of the anti-idiotype, or by its coupling to the staphylococcus-protein A (SpA), respectively.

As such anti-idiotypes which mimic structurally and functionally the antigen in the case of insulin and thyreoglobulin, cytokines (IL-1, IL2, IL-3, M-CSF, GM-CSF etc.) can be replaced by anti-idiotype ABs or their Fab/F(ab′)₂-subunits, mimicking both, structurally and functionally the cytokines. These anti-idiotypes can be used directly or as a component of the heteroconjugates described above. The advantage of the inventive drugs, described here, are the minimal side-effects, as compared with conventional anti-tumor and anti-AIDS-drugs.

Numerous studies with mono- and polyclonal heterologous ABs (e.g., OKT3 or ATG), which are used clinically in patients with kidney transplants, show that the side-effects of such a treatment are low (flu-like). Contrary to cytotoxic agents, the drugs described here show no long-term side-effects.

The eliminated lymphocytes show a complete recruitment from immature precursors within some days after the treatment has been stopped. The problem of neutralization of heterologous ABs can be circumvented.

The HIV are efficiently decomposed in RES-organs, together with the host-cells, independently of the location (nuclear DNA or cytosole). The RES-macrophages, such as the Kupffer-cells in the liver, are specialized to a quick degradation of aged (senescent) and opsonized leukocytes; the lysosomal DNAses or RNAses of the RES-macrophages are not able to discriminate between cellular and viral DNA or RNA. Even when macrophages/monocytes or, in the extreme case, the leukocytes generally are depleted, e.g., by means of the mentioned anti-leukocyte/HLe-l-AB or ALS/ALG, the treatment is less aggressive for the patient than e.g., the whole-body irradiation or the high-dosed chemotherapy in leukemia patients, because in the case of treatment with ABs, the stem cells, as well as the early precursors are preserved and lead to an early replacement of depleted mature immunocytes.

The ABs, included in the inventive drugs, described here, recognize specific surface-antigens on T- and B-cells; they are commercially available and can be produced by deposited cell lines. So, anti-CD4-AB is produced by the cell line ATCC CRL 8002; anti-CD3-AB by the cell line ATCC CRL 8001; anti-CD8-AB by the cell line ATCC CRL 8014, 8013 or 8016; anti-T10-AB by the cell line ATCC CRL 8022; anti-T11-AB by the cell line ATCC CRL 8027; anti-transferrin-receptor-AB by the cell line ATCC CRL 8021; anti-Leu15- or anti-CD11b-AB, respectively by the cell line ATCC CRL 8026 and anti-T1-AB by the cell line ATCC CRL 8000.

The present invention provides drugs for the treatment of autoimmune diseases. Autoimmune diseases are induced by autoaggressive T- and/or B-cells. They result from an impaired self-tolerance; the autologous lymphocytes “confuse” self-structures (autoantigens) with non-self antigens and induce against them a destructive cellular and/or humoral immune reaction.

The conventional treatment of autoimmune diseases by immunosuppressive agents, based on corticosteroid- or cytotoxic drugs is associated with an essential problem. Both classes of immuno-suppressants, corticosteroids (prednisone, prednisolone) and chemotherapeutic drugs (azathioprin, methotrexat, cyclophosphamide) impair only proliferating, autoaggressive cells, sparing the highly active mature enddifferentiated but non-proliferating T- and B-cells.

Therefore, the drugs, described here, based on specific ABs and their conjugates, focus to eliminate these refractory, mature, autoreactive T- and B-cells (phase I); to prevent later relapses, in addition autoantigen-specific suppressor cells have to be generated and clonally expanded in vitro or in vivo (phase II).

In the phase I, autoaggressive T- and/or B-cells, whose cell-proliferation must be considered as determined, can be accomplished by specific ABs in free or conjugated form. The conjugates are composed of AB and cytotoxin (“immunotoxin”), radionuclide or cytotoxic agents.

In the phase II, specific suppressor cells, preventing later relapses, have to be generated in vitro or in vivo. These suppressor cells must prevent or inhibit the induction and the activity of autoaggressive clones. The autoantigens—so far as they are known—represent structures that are to a large extent identical or histocompatible and therefore standardizable in the homologous system, i.e., within the same species. They are partly mimicked by the microorganism-antigens which induce, based on their homology with autoantigens, i.e., “antigenic mimicry,” autoaggressive clones, leading to the autoimmune disorder.

Since the autoaggressive T-cells as well as their inducers belong to the CD4-positive cells, the sole use of anti-CD4/T4/Leu3-AB—according to the simplest therapeutic schedule—is sufficient. A favourable side-effect is the prevention of neutralizing ABs by anti-CD4-AB. In general, neutralizing antibodies can be prevented by admixing these anti-CD4- and/or anti-CD19- or anti-CD20- or anti-CD22-ABs to antibodies or immunoconjugates, dealt with above.

Autoaggressive cells can also be impaired by combinations of anti-CD4/T4/Leu3-AB with anti-CD8/T8/Leu2-AB; anti-CD8-AB with anti-CD3-AB; further by anti-CD2/T11/Leu5-AB; by anti-CD5/T1/Leu1-AB or anti-CD45/HLe-1-AB, as well as by anti-MHC II (HLA-DR/Ia)-AB, by anti-CD25/IL-2(Tac)-receptor-AB, by anti-T9/transferrin-receptor-AB, and by anti-CD38/Leu17/T10-AB as well. These ABs can be used alone or combined. They codeplete autoaggressive cells.

Since NK-cells are also considered as auto aggressive cells by some investigators, these can be depleted by anti-CD16/Leu11- and/or anti-NKH-1/Leu19-ABs.

In autoimmune disorders, the autoantibodies often play an important role. Lymphopenia and the absolute or relative T-cell decrease, as well as the repeatedly reported selective suppressor T-cell-deficiency can originate in the pre-development of ABs, specific for autologous lymphocytes. The humoral level of the disorder is induced by autoreactive B-cells, primarily their terminally differentiated form, the plasma cells. These, on the other hand, need the “help” of T4-cells. The elimination of these plasma B-cells can be achieved by anti-CD19/Leu12- and/or anti-CD20/Leu16- and/or anti-CD22/Leu14-AB. These ABs can be combined with anti-CD4-AB. In the case that autoaggressive T-cells are present, ABs, directed against them, are used additionally.

To increase the efficacy, the ABs can be conjugated with cytotoxins, like ricin-alpha-chain, abrin, diphtheria-toxin/toxid, with cytotoxic drugs, such as doxorubicin and/or with radionuclides, such as ¹³¹⁽¹²⁵⁾I or ¹¹¹In. In addition, heteroconjugates, described in detail in association with the treatment of tumor- and AIDS-patients (see above), can be used in patients with autoimmune disorders.

These ABs and AB-combinations can be used per se or completing the conventional therapies; if integrated into the conventional therapeutical schedules, the task of these ABs is the elimination of refractory autoaggressive clones, resistant to corticosteroids or cytotoxic drugs.

In phase II, autoantigen-specific suppressor cells are generated and clonally expanded. An antigen becomes tolerogen, if the immunization is performed under immunosuppressive conditions, favoring the predominance of suppressor T-cells over helper and cytotoxic T-cells; these suppressor T-cells inhibit the immune stimulation on the cellular and humoral level (“down-regulation” of the immune response).

The purified autoantigen in a standardized form, representing the target of autoaggressive cells (e.g., the acetylcholin-receptor in myasthenia gravis or the thyreoglobulin in Hashimoto-thyreoiditis or the basic myelin-protein in MS), has to be coincubated with patient's lymphocytes for 4-6 days.

The immunosuppressive conditions of the cell culture can be maintained by cyclosporine A, by anti-HLA-DR-Fab/F(ab′)₂, by anti-CD4-Fab/F(ab′)₂, by anti-NKH-1/Leu19-AB, by corticosteroids (prednisone, methylprednisolone), by cytotoxic agents (methotrexate, azathioprine, endoxane), by PGF2, by anti-interferon, by anti-IL-2 and/or by ConA (PNA).

After the generation of autoantigen-specific suppressor cells, these can be postexpanded (in the presence of immunosuppressants). Thus, pretreated patient's lymphocytes are reinfused into the patient.

Suppressor cells can be generated directly in vivo, as well. This is accomplished by the immunosuppressive treatment of patients, predepleted in vivo of their autoaggressive T- and/or B-cells. Autoaggressive T-cells can be suppressed also by conjugates, composed of IL-2 or anti-CD25-, anti-T9- and/or anti-HLA-DR-AB and cyclosporin A or PGE2/PGE1.

Conjugates, composed of autoantigen or its anti-idiotype (Fab/F(ab′)₂-subunit) and cytotoxin and/or by conjugates, containing the autoantigen or its anti-idiotype and tolerogen (e.g., D-GL), are able to eliminate the autoaggressive T- and B-cells in a highly selective way.

Alopecia areata shows all characteristics of an autoimmune disorder. In the hair follicles, there are cell infiltrates, containing T4- and T8-cells, macrophages, dendritic cells and HLA-DR-positive epithelial cells, as well. Typically, an increased T4/T8-ratio (from 2 to 4, in some cases up to 20), as well as a strong increase of HLA-DR-positive cells can be observed. In addition, the T4/T8-ratio in peripheral blood can be elevated during the active phase of the disease. A therapy of different alopecia-forms, such as alopecia areata and alopecia generalis, can consequently be performed to some extent analogously to that in other autoimmune disorders.

The therapy is based on the same substances and mixtures (ABs and conjugates), as described above in association with “classical” autoimmune disorders, the only difference being that the autoantigen is composed of a standardized mixture of cells, cell-fragments and (3MKCl)-extracts, prepared from the hair follicles.

The autoaggressive T- and B-clones can be eliminated in a selective way by immunoconjugates/composed of autoantigen or its anti-idiotype and cytotoxin and/or by conjugates, composed of the same autoantigen or its anti-idiotype and tolerogen (D-GL).

New substances, improving vaccines, were developed. In general, the aim of vaccination is the prevention of a later infection by preimmunization with viable or prekilled microorganism (pathogen) or its extract. The vaccines induce the “immunological memory,” protecting the vaccinated person against the pathogen; this “immunological memory” is composed of “positive” and “negative” memory cells; “positive” memory cells are helper- and cytotoxic (CTL) T-cells, “negative” memory cells are suppressor T- and B-cells.

The later immune response against the pathogen depends to a large extent on the ratio of “positive” to “negative” memory cells. Consequently, the aim of every vaccination must be to delay the cogeneration of suppressor-T-cells, in order to ensure a maximal number of generated helper- and cytotoxic T-cells, as well as plasma cells; these cells have an essential impact on the “immunological memory”. This aim can be reached by the elimination of locally induced suppressor cells, as well as by substances, delaying the cogeneration of suppressor cells during the vaccination.

The vaccination can be considered as “priming” against the pathogen, deciding about the composition of memory cells, whereas the re-vaccination corresponds to the “boosting,” i.e., to the clonal postexpansion of generated subclones. Under immunostimulating conditions, the generation of suppressor cells can be delayed and herewith the induction, both of a high number of “positive” and of a low number of “negative” memory cells warranted.

The vaccination should be administered intradermally, subcutaneously or intramuscularly, to ensure an early involvement of macrophages in the immune process. The local suppressor cell-generation should be prevented or delayed by the following substances or substance-mixtures:

ABs against suppressor T-cells, such as anti-CD45R/Leu18(2H4, HB10, HB11, 3AC5)-ABs; IL-2; IL-1; IL-3; M-CSF; GM-CSF; BRM (MDP; MTP-PE . . . ); anti-PGE2; PGE2-inhibitors (e.g., indomethacin); anti-histamine (histamine-antimetabolites; blockers of the histamine-receptor); anti-corticosteroids (e.g., anti-hydrocortisone); conjugates, composed of pathogen and tolerogen (D-GL); mercaptoethanol; thioglycollate; mitogens (sub-dosed mitogenic ABs and/or mitogenic lectins); enzymes (hydrolases, such as lysozyme, neuraminidase/sialidase, hyaluronidase, special proteases and lipases); sub-dosed antibodies against the soluble T-cell receptor and against the soluble CD2/T11/Leu5 which has been described as suppressor factor, as well as against the soluble MHC II-molecule which is immunosuppressive as well (sub-dosed anti-HLA-DR/Ia 1-12-AB).

To prevent an early anti-idiotypic counterregulation, the stabilization with the conjugate, composed of idiotype or its Fab/F(ab′)₂-subunit and tolerogen (D-GL or IgG) is recommended. The same goal can be achieved by the vaccination with anti-idiotypes (favourably with their Fab/F(ab′)₂-subunits). The anti-idiotypes should be combined with a strong adjuvant (Al-hydroxid, Al-sesquioxid, Al-phosphate) and injected parenterally as described above.

The addition of high molecular weight-PEG and/or PVP directly or in combination with the adjuvant (adsorbate-vaccines) is advantageous, since these polymers facilitate the contact between immunocompetent cells and the activation of effector cells (macrophages, NK-cells).

In addition, a drug for the treatment of viral infections which are accompanied by immunosuppression (hypo- or anergy), has been developed.

It is known that infections cause immunosuppression, particularly with retroviral infections. One of them, the AIDS/ARC/LAS, has been studied in all details, including the accompanying immune depression. It has been observed, e.g., that the number of CD8-positive cells is increased significantly in patients with ARC; an increased Leu2⁺7⁺/Leu3⁺-ratio induces the relapse. The strong virus-induced immune suppression is reflected in the hypo- and anergy of patients.

The spread of the pathogenic microorganisms in the body is facilitated by the immunosuppressive constellation of the patient. Consequently, drugs that are used are not sufficiently supported by patient's immune system. Due to the critical impact of the immune suppression on the outcome of the disease, the disease-maintaining immunosuppressive constellation of the patient can be changed by (a) ABs and/or their conjugates, eliminating/coeliminating patient's suppressor cells (phase I) and (b) by an indirect in vitro preactivation or a direct in vivo reactivation of patient's effector cells (phase II).

For this purpose, the component A and B, described above, are suitable; the component B, used in the phase II, contains, however, the pathogen instead of tumor antigen or HIV.

The therapy of immune complex-associated diseases (autoimmune disorders, glomerulonephritis, chronic infections, neoplastic diseases) can be essentially improved by supporting or replacing the clinically used oral enzyme preparations by the in vivo elimination/coelimination of B-cells. This can be done (a) by anti-CD19- and/or anti-CD20- and/or anti-CD21- and/or anti-CD22-AB or their conjugates with cytotoxins (“immunotoxins”), further (b) by the combination of (a) with anti-CD4/Leu3/T4-AB or its conjugates with cytotoxins, (c) by the conjugate of the autoantigen or its anti-idiotype with the cytokine and/or with a strong tolerogen (D-GL), provided that this autoantigen is known, such as in the case of glomerulonephritis with ABs, directed against the glomerular basal membrane, (d) by the combination of the ABs and immunotoxins, cited under point (a) and (b), with ABs, directed against complement and its subunits (factors), (e) by the Fab/F(ab′)₂-subunits (a) of ABs, recognizing the Fc-receptors (both the proteinase-sensitive and/or the proteinase-resistant receptors) and/or (b) of ABs, recognizing the complement-receptor 1 (C3b-specific), receptor 2 (C3d-specific) and receptor 3 (C5a-specific). The chemotaxis and activation of tissue-destroying macrophages and (neutrophilic) granulocytes can be prevented (an example of such a Fc-receptor-recognizing AB is the CD16/Leu11a/b/c-AB), (f) by the Fc-subunit of IgG (=Fc-gamma), by which, both the binding and activation of macrophages and granulocytes can be inhibited and finally (g) by anti-alpha2macroglobulin and/or anti-alpha1-proteinase-inhibitors, inducing the early inactivation of the tissue-destroying proteases.

Induction of the inflammatory process in rheumatic diseases (rheumatic arthritis, RA) is associated with interleukins (IL-1 and IL-2), as well as with leukotriens, e.g., LTB4). IL-1 induces the secretion of PGE2 and of collagenase. LTB4 is chemotactic for macrophages. PAF (“platelet activating factor”), secreted by macrophages, is chemotactic as well. Therefore, anti-PAF-ABs, anti-PGE2-ABs, anti-LTB4-ABs, anti-IL-1-ABs, anti-IL-2-AB, collagen/tropocollagen-conjugates can be used therapeutically with cytokines and/or tolerogens (D-GL), alpha2-macroglobulin and/or alpha1-protease-inhibitor, anti-CD9(CD20, CD21, CD22)-ABs, per se or combined with anti-CD4/Leu3/T4-AB, Fc-subunit of IgG or anti-complement- or anti-complement-subunit(s)-ABs.

The system idiotype: anti-idiotype works faster and is more sensitive than the system antigen:antigen-recognizing immunocytes. The counterregulation of idiotypes by anti-idiotypes is based on idiotype-recognizing anti-idiotype-bearing CTLs and on anti-idiotypic antibodies, produced by specific B subclones; it is different from the action of conventional idiotype-bearing suppressor cells. The anti-idiotype can be considered as a high-density surface antigen or even as a pre-processed antigen (epitope) on the surface of APC (macrophage). It should be emphasized that both the activated anti-idiotype-bearing T- and B-cells are MHC II-positive. Antigen, presented in this form, is highly enriched and highly immunogenic, as compared with the nonprocessed, free antigen. By specific conjugates, immunorelevant structures on immunocytes (T4- and/or T8-cells) can be “cross-linked” and activated, to some extent independently of the APC (“intracellular/intramembranal cross-linkers”). Alternatively, the immunocompetent cells can be brought into an intimate contact by “intercellular cross-linkers”.

The present invention provides vaccines, based on anti-idiotypes and on special intracellular (intramembranal) and/or intercellular cross-linkers (heteroconjugates). Anti-idiotype-ABs or their Fab/F(ab′)₂-subunits can be used per se or conjugated as haptenic groups to strongly immunogenic carriers. It is advantageous to use as haptens (“internal mirror image”) not only one but several antigens or epitopes of the pathogen (bacterium, virus, including HIV, oncovirus). Similarly, the same anti-idiotype or its Fab/F(ab′)₂-subunit should be coupled to several different carriers.

To prevent the induction of counterregulating anti-idiotypes and consequently to increase the clonal expansion of the pathogen-recognizing idiotypes (CTL- and B-memory cells), responsible for the later pathogen-protection, pretreatment with conjugates, composed of the idiotype-Fab/F(ab′)₂-subunit of the xenogeneic, the pathogen-recognizing AB and cytotoxin can be used. Alternatively or additionally, the same idiotype or its subunit can be coupled to strong tolerogens (e.g., D-GL). The conjugates with cytotoxins induce the elimination of counterregulating anti-idiotypic T- and B-subclones, whereas the conjugates with tolerogens additionally prevent their post-recruitment.

An additional, to some extent APC (macrophage)-independent activation of T4- and/or T8-cells can be achieved by “intramembranal cross-linkers,” i.e., conjugates, composed of anti-CD3- or anti-Ti/WT31- or anti-CD2-ABs and anti-CD4- or anti-CD8-ABs (or their Fab/F(ab′)₂-subunits), respectively. The combination of these T4- or T8-activating conjugates with immunoconjugates, composed of IL-1 or Tp67 (Fab/F(ab′)₂-subunits) and anti-CD4- or anti-CD8-AB (Fab/F(ab′)₂ and/or by conjugates, composed of IL-2 and anti-CD4-or anti-CD8-AB (Fab/F(ab′)₂) is advantageous. Of special interest are also conjugates, composed of IL-1 and IL-2 or conjugates, containing Tp67 and IL-2, as well as conjugates, composed of mitogenic lectins (e.g., PHA) or their subunits and anti-CD4- or anti-CD8-AB (Fab/F(ab′)₂-subunit). An antigen (pathogen)-specific, APC-independent activation of T4- and T8-subclones (and via T4-subclones of the B-subclones) can be achieved by special conjugates, composed of antigen (fragment) or its anti-idiotype (Fab/F(ab′)₂) and anti-CD4 or anti-CD8 (Fab/F(ab′)₂). In these antigen-specific T- and B-cell activation, the mitogenic ABs (e.g., anti-CD3/Ti-AB) or mitogenic lectins (e.g., PHA) are replaced by antigen or its anti-idiotype. An additional form of the accelerated T4- and T8-activation represents the “intercellular cross-linkers”. These conjugates can be antigen-specific or non-specific. The antigen-specific conjugates are composed of antigen (or its peptide-subunit) or the corresponding anti-idiotype and anti-CD11c- or anti-CD14- or anti-CD15- or anti-CD16-AB; again, ABs can be replaced by their Fab/F(ab′)₂-subunits.

The antigen-non-specific conjugates are composed of mitogenic ABs (Fab/F(ab′)₂-subunits), such as anti-CD3-, anti-Ti/WT31-, anti-CD2-AB, or of mitogenic lectins, such as PHA (or their subunits/monomers) and of anti-CD11c- or anti-CD14- or anti-CD15- or anti-CD16 (or their subunits).

These “intercellular cross-linkers” are advantageous in the special case of the anti-HIV-vaccination, as well. In this case, the antigen-specific conjugates consist of HIV-antigens, especially env-glycopeptides (or their peptide-fragments), or the corresponding anti-idiotypes, respectively, and of anti-CD11c- or anti-CD14- or anti-CD15- or anti-CD16 (or their subunits).

The vaccine-induced expansion of disease-protective T(CTL)- and B-subclones can be further facilitated by a pre-treatment (2-7 days before the vaccination) with alkylating agents, such as endoxan, further by a conjugate, composed of pathogen (or its oligopeptidic fragment) or, alternatively, of the pathogen-mimicking anti-idiotype (Fab/F(ab′)₂-subunit), respectively, and tolerogen, such as D-GL or PEG or isologous/autologous IgG or PGE2-inhibitor; in addition, PGE2-inhibitors (indomethacin, aspirin), cimetidin and/or theophyllin (theobromin) can be added. The same carries for the anti-HIV-vaccination, as well.

The treatment with pre-synthesized “intercellular cross-linkers” can be replaced by the successive treatment with biotin-coupled mitogenic antibodies (e.g., anti-CD3- or anti-Ti/WT31-AB) or their Fab/F(ab′)₂-subunits, respectively, followed by the treatment with avidin-coupled antibodies (Fab/F(ab′)₂) of the anti-CD11 c-(anti-CD14-, anti-CD15-, anti-CD16-)type. Herewith a selective interaction of immunocompetent cells and consequently a T-cell activation can be achieved, as well. This is valid for the special case of the anti-HIV-vaccination, as well.

By preventing the counterregulation of antigen-specific ABs, the early inhibition of the clonal expansion of cytotoxic T-cells can be circumvented. This can be accomplished by (mono- and/or polyclonal) anti-B-cell-ABs or by the corresponding immunotoxins.

The clonal expansion of cytotoxic T-cells, including about 10% of IL-2-producing clones, can be increased by anti-CD4-AB and/or anti-Leu18-AB or by the corresponding immunotoxins, respectively.

In the 2nd phase of the immunization (boosting, following 6-12 days after the first immunization/priming), the strongly proliferating antigen-specific suppressor cells can be eliminated by cytotoxic drugs (alkylating agents), such as endoxan and/or by anti-CD25/Tac:cytotoxin- or IL-2:cytotoxin-conjugates.

The efficiency of the vaccines can be essentially increased if the vaccines are combined with adjuvants and/or if a technically more sophisticated step is included, consisting of a pre-separation of the peripheral blood leukocytes into macrophages, i.e., adherent cells, and into lymphocytes, followed by the incubation of macrophages with the antigen (pathogen) for 24 hours, their re-mixing with the lymphocytes and their re-infusion. The efficiency of the vaccine can further be increased by the “chessboard” vaccination (Sedlacek).

The “conventional” anti-AIDS-vaccines do not include integral parts that would be able to prevent the co-generation of suppressor subclones, responsible for the early down-regulation of the immune response. It is known that even the proliferation of the vaccinnia virus, which acts as carrier(vector)-virus in the recombinant anti-HIV-vaccine, is associated with a strong immune suppression.

The vaccines described here include components which circumvent this disadvantage of the “conventional” anti-AIDS-vaccines. They are based on conjugates of HIV-antigens (env, pol and/or gag-antigens), or their anti-idiotypes with strongly immunogenic carriers; advantageously, the 6-20 amino acids containing peptide-fragments of these vital antigens and/or the Fab/F(ab′)₂-subunits of the corresponding anti-idiotypic ABs are coupled as haptens to the same strongly immunogenic carriers. A special case is the prophylactic vaccination with the gp 120 or its peptidic fragments, respectively, as well as the vaccination with anti-CD4-AB or its Fab/F(ab′)₂-subunit. This vaccination can be essentially improved by coupling the Fab/F(ab′)₂-subunit of the anti-CD4-AB, or by conjugating peptidic fragments of gp 120 to one or, favorably, to several different, strongly immunogenic carriers.

To improve the vaccine further, these hapten:carrier-conjugates can be combined with analogously composed conjugates which recognize additional viral structures. To cut out the counterregulating anti-idiotypes before they can proliferate and consequently increase the clonal expansion of HIV-attacking cytotoxic T- and B-memory cells, we recommend the pre-treatment with conjugates, composed of idiotypes, i.e., the Fab/F(ab′)₂-subunits of ABs with specificity for the HIV-structures, and cytotoxin. Alternatively or additionally, the same idiotype or its subunit can be coupled to strong tolerogens (e.g., D-GL). The conjugates with cytotoxins lead to the elimination of the counterregulating anti-idiotypic T- and B-subclones; the conjugates with tolerogens prevent additionally their post-recruitment. An additional, partly APC(macrophage)-independent activation of T4- and/or T8-cells can be achieved by “intramembranal cross-linkers,” i.e., by conjugates, composed of anti-CD3- or anti-Ti/WT31- or anti-CD2-AB and anti-CD4- or anti-CD8-AB, respectively.

An antigen-specific T4- or T8-activation (and via T4- the B-activation) can be achieved with conjugates, composed of viral antigens (or their peptidic subunits) and/or the corresponding anti-idiotypes, respectively, and anti-CD4- or anti-CD8-AB, or their subunits.

There is an advantageous combination of these T4- or T8-activating conjugates with immunoconjugates, composed of IL-1 or IL-2 or Tp67 (Fab/F(ab′)₂-subunit) and anti-CD4- or anti-CD8-AB (Fab/F(ab′)₂). Important also are the IL-1:IL-2- and the Tp67:IL-2-heteroconjugate, as well as conjugates, composed of mitogenic lectins (e.g., PHA) or their subunits (monomers) and anti-CD4- or anti-CD8-AB (Fab/F(ab′)₂).

The following steps include a highly effective, however technically complex prophylactic vaccination: first, the adherent cells (macrophages) have to be separated from the peripheral blood; in addition, they have to be incubated in the presence of inactivated free or membrane-bound HIVs and macrophage-activators (e.g., endotoxin, gamma-interferon, MDP/MTP-PE), as well as the PGE2(PGE1)-inhibitors (indomethacin, aspirin) and/or anti-PGE2, and thereafter these HIV-structures-presenting, untreated or post-inactivated (mitomycinC, irradiation, formaldehyde/glutaraldehyde) macrophages have to be re-injected. The antigenicity of these viral structures can be essentially increased by the pre-treatment of the env-glycopeptides, especially gp 120 (gp 160) with mucopolysaccharidases (neuraminidase, lysozyme).

In bone marrow-recipients, such as total body-irradiated and/or high-dose chemotherapy-treated leukemia patients, the residual tumor-protective suppressor cells must be eliminated, analogously to the situation, described above in tumor patients. The in vitro pre-activation or in vivo re-activation of immunocompetent effector cells, preventing the proliferation of residual tumor- and (premicro)metastatic cells, as well as the explosive proliferation of bacterial and/or viral infections in the critical phase, preceding the re-establishment of patient's immune response, shows analogies to the immune re-activation, immediately following the suppressor cell-predepletion, which has been described in detail in tumor- and AIDS-patients (see above).

Due to the non-specific pre-depletion of immunocompetent lymphocytes as a consequence of the total body-irradiation or high-dose chemotherapy in bone marrow-recipients, both the amount of ABs and immune potentiators, as well as of the re-activated effector cells can be reduced considerably.

In histoincompatible bone marrow-donors, the recipient-specific alloreactive T-cells can be pre-depleted, prior to their infusion into the recipient, by the pre-activation of these cells in a kind of MLC/MLR-assay, followed by their selective elimination with immunotoxins, based (a) on specific ABs, recognizing the activated subpopulation among the donor's T-cells, or (b) on IL-2 (i.e., cytotoxin:IL-2-conjugates).

Alternatively, allospecific suppressor cells can be pre-generated in vitro and admixed to the donor bone marrow-cells prior to their infusion into the recipient. This in vitro pregeneration of allospecific suppressor cells is performed in an MLC/MLR-assay, under strongly immunosuppressive assay-conditions.

The innovative drugs, ensuring the in vivo depletion of tumor-protecting suppressor cells, as well as the re-activation of tumoricidal effector cells, correspond to those described in detail in tumor- and AIDS-patients.

The in vitro pre-depletion of alloreactive T-subclones can be achieved by anti-CD25/IL-2-receptor-AB, anti-CD45-AB and/or anti-HLA-DR-AB (and complement) or by the corresponding immunotoxins. These ABs can be admixed to the mixture of donor- and recipient lymphocytes that have been coincubated for 4-5 days; by these ABs, the alloreactivated—and only these—T-cells are depleted.

The allospecific suppressor T-cells can be pre-generated by a 6-10-day coincubation of donor and recipient lymphocytes in a medium, containing corticosteroids, such as by hydrocortisone, prednisone, methylprednisolone, further immunosuppressive cytotoxic agents (azathioprine), cyclosporine A, PGE2 (PGE1), anti-HLA-DR-AB, anti-CD4-AB, ConA and/or anti-IL-2 (anti-IL-1). These allospecific suppressor cells have to be admixed to donor's bone marrow prior to its infusion into the recipient.

The autologous bone marrow-transplantation in patients with solid tumors can be improved by CK-2-AB or by immunotoxins, based on this CK-2-AB. Autologous lymphocytes, as well as LAK- and TIL-cells can be freed (purged) of contaminating tumor cells by such cytokeratin-recognizing ABs and corresponding immunotoxins.

There exist a series of additional conjugates, used per se or as a component of the novel drugs, described here, which show immunostimulating and/or immunotherapeutical effect and can therefore be considered as an integral part of the therapeutical approaches, discussed above. They intervene actively in the immunoregulation and have a positive impact on a series of diseases. Some of them are able to improve essentially the success of organ (kidney), and/or bone marrow-transplantations.

A variant of such compounds are immunoconjugates, composed of the target cell-recognizing ABs and enzymes, activating cytotoxic drugs locally (target cells:tumor cells, (retro)virally, e.g., HIV-infected cells, leukocyte-subpopulations). The enzymes, as an integral part of specific conjugates, are “fixed” to the tumor cells and activate in situ/locally the cytotoxic agent by transforming the inactive (prodrug-) form into the active (drug-)form (example: oral, in situ activable cytotoxic drugs). The same principle is effective in the elimination of leukocyte subclasses, e.g., suppressor T-cells. In this case, the anti-tumor-ABs in immunoconjugates have to be replaced by anti-leukocyte-ABs or, alternatively, by cytokines (monokines, lymphokines).

A further variant of such compounds includes immunoconjugates, composed of target cell-recognizing ABs and enzymes, cleaving essential cell metabolites in situ/locally, depriving in this way the target cell of vital substrates (“starving out” of the target cells) (target cells:tumor cells, (retro)virally, e.g., HIV-infected cells, leukocyte-subpopulations). This immunoconjugate-class can replace or support the classical immunotoxins; thus, the eventual problems, associated with the RES-toxicity of immunotoxins can be circumvented. The principle of these novel immunoconjugates is the local impoverishment of target cells (tumor-cells, leukocyte-subpopulations) concerning the essential cell substrates. Examples are immunoconjugates, composed of the enzymes asparaginase, glutaminase, arginase, methioninase, beta-tyrosinase, phenylalanin-ammoniumlyase, xanthin-oxidase or folic acid-catabolizing enzymes (carboxypeptidase G) and ABs, recognizing membrane-structures on the target cells, such as tumor-antigens, differentiation-antigens etc.

If a selective killing of leukocyte-subpopulations is intended, the cell metabolite/substrate-catabolizing enzymes can be coupled to cytokines (monokines, lymphokines) instead of Abs.

Generally, this immunoconjugate-class consists of enzymes which deprive the target cell of metabolic precursors and of corresponding ABs (or their Fab/F(ab′)₂-subunits) which fix these enzymes exclusively to the surface of the target cells.

The “enzymatic therapy,” based on a systemic administration of the enzyme asparaginase or arginase (per se, not as conjugate), is non-selective and non-localized, the amount of enzyme(s), needed for this kind of therapy being high. The action of enzyme-conjugates with target cell-recognizing ABs or their Fab/F(ab′)₂-subunits, on the contrary, is localized (confined to target cells) and therefore selective.

A further variant represents immunoconjugates, composed of target cell-recognizing ABs and enzymes, activating target cells, e.g., leukocyte-subpopulations in a direct and selective way. The basic principle of this immunoconjugate-class is the “fixing” of target cell-activating enzymes to the surface of the same. Detailed experiments have shown that hydrolases (proteinases, lipases, mucopolysaccharidases) activate cells within some minutes and that immunocompetent cells themselves use this principle to amplify their activity. The advantage of these enzymes and of immunoconjugates, containing the enzymes, is their effect, both on enzyme-loaded target cells, as well as on neighboring cells that have not been “reached” by the immunoconjugate. For the same reason, the radionuclide-containing immunoconjugates are superior to cytotoxin (ricin or doxorubicin)-containing ones.

The novel class of immunoconjugates, described here, is based on a hydrolytic enzyme (proteinase, such as trypsin, chymotrypsin, papain, bromelain; lipases, mucopolysaccharidases, such as sialidase/neuraminidase/RDE of lysozyme) which is coupled to a target cell-recognizing AB. The ABs can be replaced by cytokines (monokines, lymphokines), since the affinity of cytokines for their receptors is generally even higher than the affinity of ABs for the specific antigens.

Leukocyte-subpopulations can further be activated selectively by conjugates, composed of target cell-recognizing ABs and target cell-activating components, as e.g., immunoconjugates, composed of immunopotentiators, stimulating macrophages/monocytes, NK-cells or T-cells, and target cell-recognizing ABs, such as conjugates, consisting of gamma-interferon, lysolecithin, endotoxin or MDP (MTP-PE), and anti-M1(M3, M5, CR1)-AK, or conjugates, composed of statolon or phorbol ester and anti-CS2(CD5)-ABs. This immunoconjugate-class mediates the fixation of cell-activating molecules directly to the surface of target cells; their dose can therefore be kept relatively low.

In addition, tumor cells or (retro)virally (e.g., HIV-)infected target cells can be attacked by conjugates, composed of target cell-recognizing ABs and cytolytic cytokines, such as TNF or lymphotoxin. Conjugates, consisting of TNF or lymphotoxin and anti-tumor-ABs and anti-gp-120-ABs are of special interest. By this class of conjugates, the cytotoxic (tumoricidal) effector substances can be directed selectively to the target cells; the side-effects can be strongly reduced by the dose-decrease.

The antigen-specific T-cell-activation can be achieved by mixtures, containing such mitogenic ABs which do not interfere sterically with the T4:APC-interaction (e.g., anti-CD5/T1- or anti-CD2/T11-AB) and Tp67 or IL-1. By these ABs or, more advantageously, their Fab/F(ab′)₂-subunits, T-cells can be brought into a “pre-activated” (“stand-by”) state and can afterwards be activated in a selective manner by the specific antigen.

Of special importance are conjugates, composed of target cell-specific ABs and effector cell (macrophage/monocyte-, NK- and CTL-)-specific immunopotentiators (BRM, “biological response modifier”). Examples are conjugates, composed of anti-CD11c(CD14-, CD15-)-ABs and MDP (or MTP-PE or thioglycol) which activate macrophages specifically, at BRM-doses, being essentially lower than those, used in conventional therapeutical approaches; consequently, the toxic side-effects of BRMs are considerably reduced.

An interesting example are conjugates, composed of anti-T-cell-ABs and 2-mercaptoethanol, since 2-mercaptoethanol is able to replace partially the macrophage(factors) during the (polyclonal) T-cell activation. The BRM-application in the form of (hetero)conjugates shows essential advantages in comparison to the liposome-encapsulated BRMs, since liposome-encapsulated BRMs are exposed to lysosomal enzymes, following their internalization in macrophages, leading to their inactivation or disintegration. In addition, heteroconjugates are more mobile in the body than liposomes which are not able to leave the bloodstream.

Examples of immunopotentiators are tilorone, propane-diamine, mercaptoalkylamine, BL-20803, MA-56, AET, U-25/166, PMA (phorbolmyristatacetate), cimetidine, quinacrine, bacterial extracts, lipopolysaccharides, lipid A, endotoxin, toxoids, plant-extracts (lentinan, pachymaran, caboxymethylpachymaran), KLH, laetile, levamisole, zymosan, TNP-KLH, statolone, paran-copolymers (maleinic acid-divinylether-copolymers), COAM, levan, oxazolon, HiuI and waxC.

Of special interest are conjugates, composed of ABs, recognizing target cells (macrophages, T-cells, B-cells, NK-cells) and specific cytokines (monokines, lymphokines). So, anti-CD11c/CD14/CD15-ABs, coupled to lymphokines, such as MIF (“migration inhibition factor”), MAF (“macrophage activation factors”), M-CSF (“macrophage colony stimulating factor”), IL-3 and gamma-interferon (see above), respectively, are able to transfer these macrophage-stimulating cytokines in a highly selective way to the “right address,” i.e., target cells.

Analogously, anti-B-cell-Mabs can be coupled to lymphokines, such as BCGF (“B-cell growth factor”) or BCDF (“B-cell differentiation factor”), and combined with conjugates, composed of the same Mabs and IL-1, IL-2 and/or gamma-IFN.

The therapy with LAK (“lymphokine-activated killer-cells) and IL-2 can be improved by the compounds described herein.

The LAK/IL-2-therapy, developed by S. A. Rosenberg et al. has the disadvantage of the in vitro coactivation and clonal coexpansion of suppressor cells, as well as the disadvantage of their reinfusion into a patient whose preexisting suppressor cells had not been predepleted. Consequently, the number of needed LAK-cells must be enormous to overcome the resistance of the residual suppressor cells. Under these conditions the later relapses are preprogrammed.

To improve this situation, the cogeneration of suppressor cells during the in vitro generation of LAK-cells should be prevented by anti-Leu18-AB or by a mixture of the ½ cytolytic dose of anti-Leu8-Mab and the ½ cytolytic dose of anti-Leu18-Mab, or alternatively, by the 1/3 cytolytic doses of anti-Leu3-Mab and anti-Leu8-Mab and anti-Leu18-Mab, combined with IL-2, anti-PGE2 (anti-PGE1) and/or PG-inhibitors (indomethacin, aspirin). More efficient than Mabs which must be added together with complement, are the corresponding Mab-based immunotoxins.

Since LAK-cells consist of up to 90% of NK-cells, the cogeneration of suppressor-T-cells can be prevented by the depletion of the CD3-positive T-cell-fraction by mono- and/or polyclonal panT-specific ABs (e.g., anti-CD3-AB; ATG). Alternatively, the CD4-positive T-cells (including inducer-suppressor cells) or the CD8-positive T-cells (including effector-suppressor cells) can be eliminated. Again, the corresponding immunotoxins are more efficient than the antibodies per se.

The inactivation of radiosensitive, Leu3⁺8⁺18⁺-inducer-suppressor cells by the preirradiation of the in vitro cell culture can further increase the efficiency of the suppressor cell-elimination; the same is true with the addition of lysosomotropic substances, such as monensin, chloroquine and/or NH₄Cl. The possible contamination by tumor cells can be (partly) eliminated by CK-2-ABs (or by the corresponding immunotoxin).

Therapy with TIL (“tumor infiltrating lymphocytes”) and IL-2 (according to S. A. Rosenberg et al.) can be improved by special additives as well. Later relapses could be reduced, if, analogously to the LAK/IL-2-therapy, the cogeneration of suppressor-subclones in vitro would be inhibited and the cyclophosphamide in vivo would be combined with Mabs or, advantageously, the corresponding immunoconjugates, eliminating/coeliminating resting and memory suppressor cells being responsible for later relapses. This can be achieved by the Mabs and their cocktails, described above, as well as by the pre-irradiation of radiosensitive inducer-suppressor cells already mentioned. The coelimination of non-proliferating, end-differentiated resting and/or memory-effector-suppressor cells can be accomplished by the combination of cytotoxic drugs (e.g., cyclophosphamide) and mono- or polyclonal ABs (better: immunotoxins), recognizing the CD3-positive T-cells or their CD8-positive subpopulation.

The therapeutic approach, first described by A. Fauci, being closely related to the treatment of bone marrow recipients (total body irradiation and/or high dose chemotherapy) could be essentially improved by combining the whole body-irradiation with a post-treatment with mono- or polyclonal ABs (or immunotoxins) and reconstitution with in vitro pregenerated or in vivo reactivated virucidal effector cells. This can be achieved by substances and cocktails, described in association with tumor patients and organ graft-recipients.

A combination of the cutaneous pre-stabilization and in situ (intratumoral) immunization with the common antigen (hapten) is of special interest in the treatment of bladder cancer. The principle is the clonal preexpansion of antigen- or hapten-specific T-cells (CTLs, T_{DTH}/T_{D}). These T-cells occur in increased numbers and in preactivated state during the following intratumoral immunization (with the same antigen or hapten). As example, TNBS can be quoted: first, a cutaneous prestabilization is accomplished, followed by the bladder instillation of the same substance (TNBS); by this procedure, the pregenerated TNBS-specific T_{DTH}/T_{D}-cells migrate to the bladder to kill tumor cells, bearing the TNB-haptenic group on their surface. The mechanism is the prompt binding of substances such as TNBS to autologous proteins (e.g., cutaneous proteins) and cell membranes (e.g., of malignant bladder cells). By the strongly immunogenic haptenic groups (e.g., TNB-group), the T_{DTH}/T_{D}-cells, i.e., CD4-positive T-cells with cytotoxic properties, involved in the DTH-reaction, are induced. This class of substances comprises all strongly haptenizing compounds, such as DNCB, TNCB, DNBB, TNBB, picrylchloride, as well as TNBS (trinitrobenzol-sulfonic acid), the last cited being of special interest because of its water-solubility.

Alternatively, ABs (Mabs and Mab-mixtures), as well as their Fab/F(ab′)₂-subunits, binding to the cell surface (first of cutaneous cells, later of tumor cells), further lectins and protectins or their subunits (monomers), binding to the monosaccharide-units of the skin and later of tumor cells, and finally non-pathogenic viruses (e.g., vaccinia-virus, Newcastle-disease-virus, influenza-virus), can be used for the haptenization of target cells. Humoral suppressor factors can have an enormous impact on the immune response; therefore they have to be neutralized.

Most in vitro assays are performed in the absence of patient serum and are consequently associated with inaccuracies, due to the unknown contribution of humoral suppressor factors. The in vitro determined cell activity is mostly higher than the real in vivo cell activity. Therefore, it is important to measure the plasma level of soluble suppressor factors in addition to the cellular activity.

Although the particular suppressor factors are not known, they must be expected among the immunorelevant membrane-structures, following their shedding into the plasma. To this class of suppressor factors belong soluble IL-2-receptors, soluble MHC I- and MHC II-molecules, further soluble CD4- and CD8-structures, plasma-prostaglandins, especially PGE2 and PGE1, plasma-corticosteroids, soluble CD2-molecules, soluble T9-fraction, soluble beta2-microglobulin, as well as soluble receptors for cytokines (monokines and lymphokines) in general (e.g., receptors for IL-1, IL-2, M-CSF, GM-CSF).

By the titration of these soluble immunosuppressive factors with corresponding ABs, immunosuppressive influence of patient plasma could be neutralized. In the case of soluble cytokine(including interferon)-receptors, the titration with basic cytokines (instead of specific antibodies) could provide the basic data about the actual concentration of the corresponding solubilized receptors in patient plasma.

For both, the diagnostic and therapeutic use, ABs, neutralizing the following soluble factors, can be used: IL-2-receptor, MHC I, MHC II, CD4, CD8, CD2, T9, beta2-microglobulin, prostaglandins and/or corticosteroids (hydrocortison); in addition, the neutralization of corresponding solubilized receptors can be achieved by the cytokines IL-1, IL-3, (IL-2), M-CSF, GM-CSF and by the interferons alpha, beta and gamma, respectively.

A highly specific and at the same time immunosuppression-free elimination of the tumor- or (retro)virally (e.g., HIV-)infected cells can be achieved by anti-tumor (retroviral)-ABs or the corresponding immunotoxins (eliminating tumor or virally infected target cells, and anti-idiotypic ABs mimicking tumor or retroviral antigens); alternatively, immunotoxins, composed of anti-idiotypic-Fab/F(ab′)₂- subunits and cytotoxin, and/or of anti-idiotypic-Fab/F(ab′)₂ and tolerogen (D-GL) (anti-idiotypes in the immunotoxin can be replaced by corresponding antigens or their fragments). To prevent the neutralization of anti-tumor(anti-retroviral)-ABs by the specific anti-idiotypes or their immunoconjugates, the last ones must be injected in intervals of several days. Anti-idiotypes can be replaced by lymphocytes, plasma or blood of a person B, which has been preimmunized with the leukocytes or with the blood of the patient A.

Control with mono- and/or polyclonal ABs or the corresponding immunoconjugates achievable temporary immunoincompetence is associated with essential advantages. The T-cell-depletion terminates a pathological state, caused by the immune dysfunction of the patient and opens, due to the low resistance of the residual immunocompetent cells, novel and broad ways for the establishment of disease-preventing constellations, which can be achieved by relatively low numbers of in vitro pregenerated (specifically “tailored”) or in vivo reactivated immunocytes.

To achieve a highly efficaceous elimination/coelimination of a particular functional leukocyte-subpopulation (e.g., of allo-aggressive T-cells in the case of organ transplantation, or of tumor-specific suppressor cells in the case of neoplastic diseases, or of autoaggressive lymphocytes in the case of autoimmune disorders), the function- or antigen-selective ABs or immunoconjugates have to be combined with those ABs which eliminate mature T- and/or B-cells in general; in the case of substances which deplete T- and/or B-cells in a non-specific way, two classes can be distinguished: (a) (mono- or polyclonal) ABs and (b) cytotoxic substances, such as cylophosphamide.

The aim of this combination is an essentially increased efficiency, as well as a reduced resistance against the specifically “tailored” immunocytes to be reinfused. Therefore the combination of panT-specific monoclonal ABs, such as anti-CD3-, anti-CD5- and anti-CD2-Mab, or of polyclonal ABs, such as ATG/ATS and ALG/ALS, with anti-suppressor- or anti-idiotypic ABs in patients with neoplastic and retroviral diseases, or in the case of organ transplantations - with anti-CD4- and anti-CD8-ABS, respectively, or finally - in the case of autoimmune disorders - with anti-CD25/Tac-receptor-AB and IL-2:cytotoxin-conjugates, is recommended.

In addition, combined preparations, composed of these ABs, directed against suppressor-precursors, and suppressor cell-impairing substances, such as indomethacin, cimeditine etc., can be administered to “stabilize” the interphase between phase I and phase II, though this “stabilization” can normally be omitted.

The immunosuppressive cytotoxic drugs in the aforementioned preparations can be replaced with non-suppressive anti-tumor- or anti-HIV-ABs. Herewith the tumor- or HIV-protective idiotype-bearing T- and B-subclones are eliminated by anti-idiotypic-ABs or inactivated by their subunits. To accomplish this, the anti-idiotypic-ABs, eliminating idiotype-bearing T- and B-cells, can be combined with anti-tumor- or anti-HIV-ABs. A time interval of 10-30 days should be foreseen between the anti-idiotypic-treatment and the anti-tumor-AB-treatment.

An additional procedure in the treatment of neoplastic, (retro)viral and autoimmune diseases is the elimination of B-cells. Contrary to the situation in normal bacterial and viral infections, the role of specific ABs in retroviral infections, including HIV, in persistant/chronic infections in general, in patients with tumors as well as those with autoimmune disorders is problematic. The ABs in patients with persistent/chronic diseases, which escape the immunological defense, lead to a specific, as well as generalized immunosuppression, due to the masking of the relevant antigens on the surface of target cells and/or specific activation of TG-suppressor cells by IgG and IgG-based immune complexes, as well as due to the non-specific suppression of tumoricidal NK-cells and macrophages.

Therefore it is advantageous to prevent the generation of specific ABs by antigen-specific plasma (B)-cells in situations, such as that, immediately following the removal of primary tumor or that, following the total body-irradiation of bone-marrow-recipients. The same concerns the critical phase, immediately following the elimination of (potential) HIV-host cells and HIV-protecting suppressor cells, as described above.

An antigen-specific or generalized B-cell-depletion is, however, able to induce a humoral and mediately a cellular immune derepression, per se or in combination with other treatments, described above, in immunocompromized patients, i.e., patients with neoplastic and (retro)viral diseases, including AIDS/ARC/LAS.

The non-specific B-cell-elimination can be performed with anti-CD19-, anti-CD20-, anti-CD21- and/or anti-CD22-AB, or the corresponding immunotoxins, respectively, per se or combined with anti-CD4/Leu3/T4-AB, anti-PGE2-AB, antiarachidonic acid-AB, PG-inhibitors (indomethacin) and/or PL4 (platelet factor 4).

The tumor-specific B-cell-elimination/coelimination can be accomplished with conjugates, composed of tumor-antigen or the corresponding anti-idiotype and cytotoxin, per se or combined with conjugates, consisting of the same tumor-antigen or anti-idiotype (or Fab/F(ab′)₂-subunit, respectively) and strong tolerogen (e.g., D-GL).

In patients with neoplastic, (retro)viral (including HIV-) and autoimmune diseases, a special approach is associated with a T-cell-depletion, since their role is similar to that of B-cells, problematic to negative in situations, such as persistent diseases (retroviral, chronic, neoplastic and autoimmune diseases). The reason is specific and in advanced disease - a generalized immunosuppression, caused by suppressor-T-cells and their secretion products; the humoral suppression is based on soluble suppressor factors as well as on immune complexes (especially of the IgG-subclass), discussed in association with B-cells. Therefore, T-cells should be generally “kept away” in the critical phase, following the removal of tumor mass and the whole body-irradiation in bone marrow-recipients (leukemia-patients).

Analogous to the B-cells, a T-cell-depletion per se or combined with a B-cell-depletion could introduce an essential step, de-blocking of patient's immunosuppressive state. This can be achieved by anti-CD3/Leu4/T3-, anti-CD5/Leu1/T1- and/or anti-CD2/Leu5/T11-Mab or the corresponding immunotoxins, and/or by anti-thymocyte-serum (ATS) or anti-thymocyte-globulins (ATG), eventually combined with anti-CD19(CD20, CD21, CD22)-ABs.

Animal experiments show that viral infections (LCM-virus) can be held unter control, both, after the elimination of T4 (L3T4), as well as after the depletion of T8 (Lyt2)-T-cells. Moreover, uncoupling of T4- and T8-cells can have a positive impact on the immune response. This principle can be partially used in the treatment of neoplastic diseases as well. For this purpose, anti-CD4/Leu3/T4-AB or anti-CD8/Leu2/T8(T5)-AB, per se or combined with anti-CD19(CD20, CD21, CD22)-AB can be used. In addition, a simultaneous blocking of CD4-positive inducer-suppressor cells, responsible for the induction of cellular immune suppression and the T4-dependent B-cells, responsible for the humoral immune suppression, can be achieved by the simple depletion of T4-helper/inducer cells in patients with neoplastic, (retro)viral (including HIV-) and autoimmune diseases. By the elimination (depletion) of T4-cells, the cellular and the humoral immune response to the tumor-antigen can be achieved. A radical elimination of T-cells and their replacement by non-specific effector cells (NK-cells, macrophages) can be advantageous in some patients with neoplastic and retroviral diseases, because of the potential danger of immunosuppressive impulses. So, in vitro preactivated or in vivo preactivated effector cells (LAK-cells) in tumor patients—immediately following the removal of primary tumor - as well as in total body-irradiated leukemia patients, can be effective even in the absence of T-cells.

The depletion of potentially immunosuppressive cells can be achieved, e.g., by anti-CD4/Leu3/T4-ABs, per se or combined with anti-CD19(CD20, CD21, CD22)-ABs and their immunoconjugates with cytotoxins.

The de-blocking of the depressed immune system can be performed by the uncoupling of CD4-positive helper/inducer T-cells from the CD8-positive suppressor/cytotoxic T-cell-subset; this can be combined with a simultaneous elimination of the humoral immune response (B-cell-depletion).

Too fast and non-equilibrated cell growth can cause cell death, a principle which has been used practically in the case of the “Agent Orange”. Consequently, the immunoconjugates, composed of anti-tumor-Mabs and substances, such as auxin, gibberelins, hydroxynaphthylamines, as well as the growth factors, can be used for the elimination of tumor-cells. Locally, such conjugates can be used even in a free form. When coupled to Mabs, these substances can be dosed lower and show essentially reduced side-effects.

The efficacy of cytotoxic agents can in general be increased by mitose-inducing factors (transformation of the G₀- into the G1-phase). Analogously, substances or conjugates, described above, inducing a non-equilibrated, uncontrolled cell growth, can reduce the part of resting (G₀)-tumor cells, being refractory to the treatment with cytotoxic drugs. Therefore, the use of immunoconjugates, composed of substances, such as auxins, gibberelins, phytohormons and growth hormones and antitumor-ABs or, alternatively, local, sub-dosed use of these substances per se, both followed by chemo(radio)therapy, can bring essential therapeutical advantages.

Suppressor cells can be activated selectively by histamine. Consequently, the histamine-induced activation can be prevented by anti-histaminics and/or ABs, recognizing the histamine-receptor. Therefore, the activity of suppressor cells can additionally be inhibited by anti-histaminics and/or anti-histamine receptor-ABs.

Suppressor-T-cells have receptors for PNA. Therefore, they are vulnerable by PNA-cytotoxin-conjugates and can be eliminated by immunotoxins, composed of PNA and cytotoxin(s).

The activation of pre-existing suppressor cells with IL-2, followed by (a) their elimination by cytotoxic agents (endoxan) and/or anti-IL-2-receptor-Mab and/or IL-2-cytotoxin-conjugates, respectively, and (b) by the post-expansion of non-suppressor cells with IL-2, is an additional therapeutical procedure. The preactivation of preexisting suppressor cells can be achieved by IL-2 alone or in combination with ConA and/or PNA. The elimination of preactivated suppressor cells can be performed (a) with anti-CD25- or anti-T9- or anti-Leu17-AB or, alternatively, (b) with IL-2:cytotoxin- and transferrin:cytotoxin-conjugates or (c) with cytotoxic drugs (endoxan), respectively. Moreover, the post-activation of non-suppressor cells can be accomplished with IL-2, further with different immunopotentiators and/or with mitogens.

Activated T-cells can be eliminated by anti-CD25-, anti-transferrin-receptor-, further by anti-HLA-DR-ABs, by immunotoxins, as well as by anti-Leu17-AB or its immunotoxin.

A further variant can be considered for the bone marrow-grafts, consisting of an in vitro pre-separation of macrophages/monocytes, based on their adhesion-property, followed by their admixing to the T- and B-cell-predepleted donor bone marrow. In this special case of the bone marrow-enrichment with immunocompetent cells, macrophages/monocytes and NK-cells are the only mature/immunocompetent cells. This constellation prevents the tolerance-reinduction to residual tumor(leukemia)cells to a high degree. The admixed macrophages and/or NK-cells can be pre-activated.

In the case of autoimmune disorders, person B can be sensibilized/immunized (a) with the blood, (b) with the lymphocytes or (c) with the plasma of the patient A, followed by the reinjection of (a) the blood, (b) the lymphocytes or (c) the plasma of person B into the patient A. Herewith, the clonally preexpanded autoaggressive clones (T- and B-cells) of the patient can be selectively impaired/eliminated.

In patients with bladder tumor, allogeneic or xenogeneic erythrocytes can be pre-treated with strongly immunogenic haptens (e.g., TNBS) and used both (a) for the stabilization (in a kind of cutaneous test) and (b) for the later bladder-instillation.

With the instillation of effector cell-stimulating enzymes into the bladder, the tumor-localized effectors (macrophages, NK-cells, TILs) can be activated directly in situ.

What is claimed is:

1. A method for treating cancer in a subject, comprising:
(a) eliminating suppressor cells in the subject;
(b) preactivating lymphocytes from the subject ex vivo; and
(c) injecting the preactivated lymphocytes into the subject, thereby treating the cancer.

2. The method of claim 1, wherein the suppressor cells are eliminated in a specific way.

3. The method of claim 1, wherein the suppressor cells are eliminated in a non-specific way.

4. The method of claim 1, wherein the suppressor cells are eliminated by contacting the suppressor cells with antibodies selected from the group consisting of (a) antibodies specific for CD-8 positive suppressor cells, (b) antibodies specific for inducer-suppressor cells, (c) antibodies specific for transducer-suppressor cells, (d) antibodies specific for a subpopulation of radiosensitive suppressor cells, (e) antibodies specific for immunocompetent (pan)-T cells, (f) antibodies specific for (pan)-leukocytes, and (g) polyclonal/polyvalent antibodies or globulins directed against T-cells and thymocytes or lymphocytes.

5. The method of claim 1, wherein the suppressor cells are eliminated by contacting the suppressor cells with antibodies selected from the group consisting of T cell-specific monoclonal antibodies and T cell-specific polyclonal antibodies.

6. The method of claim 3, wherein the antibodies are selected from the group consisting of anti-CD8, anti-CD1, anti-CD3, anti-CD5, anti-CD2 and anti-pan T-antibodies.

7. The method of claim 1, wherein the suppressor cells are eliminated by contacting the suppressor cells with cyclophosphamide and monoclonal or polyclonal antibodies recognizing the suppressor cells.

8. The method of claim 1, wherein the suppressor cells are eliminated by contacting the suppressor cells with cyclophosphamide and CD-3 or CD-8 positive T-cells.

9. The method of claim 1, wherein the lymphocytes of the subject are preactivated by contacting the lymphocytes ex vivo with tumor cells of the subject.
